(19) United States
(12) Patent Application Publication
(10) Pub. No.: US 2014/0224248 A1
(43) Pub. Date: Aug. 14, 2014

(54) AEROSOL DELIVERY APPARATUS

(71) Applicants: David A. Edwards, Boston, MA (US); Jonathan Jacques Kamler, Jamaica, NY (US)

(72) Inventors: David A. Edwards, Boston, MA (US); Jonathan Jacques Kamler, Jamaica, NY (US)

(73) Assignee: AeroDesigns, Inc., Cambridge, MA (US)

(21) Appl. No.: 14/115,941

(22) PCT Filed: May 11, 2012
(86) PCT No.: PCT/US2012/037552

§ 371 Date: Feb. 25, 2014

Related U.S. Application Data
(60) Provisional application No. 61/484,899, filed on May 11, 2011.

(51) Int. Cl. A61M 15/00

(52) U.S. Cl. CPC A61M 15/0008; A61M 15/0021
USPC 128/203.15

(57) ABSTRACT

Aerosolized particles in a determined particle size range are suspended in the air and deposited in the mouth without easily entering into the respiratory tract. An apparatus incorporating an aerosol generating device and particles can allow for the aerosolization of the particles and the delivery thereof in a manner suitable for inhalation or deposition and subsequent ingestion. The particle delivery apparatus represents a novel means for delivering food to the mouths of humans and animals. Indeed, the apparatus of the invention is designed to produce, transport, and direct aerosolized particles (e.g., food particles) in a determined size range, suspended in air, to be deposited in the mouth without substantial exposure or entry into the respiratory tract.

### FIELD OF THE INVENTION

The invention relates generally to aerosolized particles and apparatus for the containment, aerosolization, and/or delivery thereof.

### BACKGROUND

Previous researchers have demonstrated that aerosol particles can be used to deliver substances to various parts of the body. Certain designs have been proposed for utilizing these particles for drug delivery.

### SUMMARY

Light, consumable particles can be drawn into a user's mouth for deposition on surfaces of the mouth for consumption through transdermal surfaces and/or through the digestive tract (e.g., ingestion via intake into the stomach and gastrointestinal tract by means of enteral administration). However, when consuming particles that are sufficiently light to be drawn into a user's mouth by inhalation or exhalation, one must address the risk of those particles reaching the back of the mouth or lungs and causing coughing or other adverse events, especially when the goal is, for example, to provide taste, nourishment, dietary supplementation, and/or medicinal delivery, involving the mouth, tongue, etc.

Therefore, approaches to deliver materials to the mouth via the airborne route have largely (if not exclusively) focused on directed, non-breath-actuated delivery, where the force of the air current and size of the particles are such that particle trajectories are primarily limited to within the mouth.

We have developed an approach by which a casual or forced breathing maneuver (such as normal inhalation or exhalation) can lead to the delivery of food, drink, medicinal and/or various other particles to the mouth, in which the transport of these particles with the flowing air, to the back of the throat and to the lungs, is limited. By controlling the inertia and gravity of the particles (e.g., food particles), and by directing deposition forces, we can focus delivery of the particles towards surfaces of the mouth, not reaching the back of the throat and lungs.

A variety of forces can be used to generate the aerosol and/or cause it to move through the apparatus and be delivered. These include a user's inhalation/exhalation, aspiration/expiration, shaking or vibration forces, and/or external power sources (e.g., compressed air, electric fans, motors, etc.).

For some embodiments, there are two practical aspects to our approach:
1. Particle size is important to the delivery system, namely that the particles need to be small enough to remain airborne during casual breathing, but large enough to be directed and deposited primarily in the mouth while limiting coughing, throat and lung deposition, or other adverse situations.
2. At the same time, typical pathways of aerosol particles through the device and out of the mouthpiece are directed to varying degrees away from the back of the throat.

In some embodiments, the combination of appropriate particle size and device-directed air pathway leads to the particles (e.g., food particles) being deposited primarily in the mouth (and onto the tongue, palate, etc.) rather than at the back of the throat or further into the respiratory tract. In some embodiments, a “sipping” or “sucking” maneuver, in which air is directed into the mouth and not simultaneously flowing into the lungs, substantially eliminates deposition in regions of the respiratory tract beyond the mouth.

A particle delivery apparatus can include features, devices, or elements for containing or receiving aerosolizable particles, and a fluid flow passage extending between an inlet and an outlet. In some embodiments, the apparatus is intended to deliver a consumable aerosolizable product to surfaces within a consumer's mouth.

The design of the apparatus—including the shapes, sizes, and orientations of its various elements—may have significant impact on a consumable product, including its aerosolization, its flow through sections of the apparatus, and its emission from the apparatus. The apparatus design parameters thus determine, at least in part, the effectiveness of the system overall in delivering a desired substance to a consumer.

In different embodiments, fluid flow passages of the apparatus can be designed with different physical parameters, for example:
1) Different lengths (e.g., aerosol flow path lengths);
2) Different tortuosities (e.g., flow path complexities);
3) Different geometries (e.g., inlet or outlet cross-sectional areas); and/or
4) Different orientations (e.g., positions of inlets and outlets relative to each other, the user, or gravity).

These exemplary physical design differences, among others, can affect fluid flow properties, such as:
1) Typical fluid resistances or pressure drops across sections of the apparatus (e.g., a pressure drop over the consumable product, which gives rise to its aerosolization);
2) Typical rates (e.g., accelerations, velocities) or time durations, in sections of the apparatus or upon emission, of particles or fluid (e.g., the time for an aerosol to displace from an inlet to an outlet, or the velocity it has upon emission);
3) Typical aerosol properties, including size, shape, orientation, particle concentration, particle-size distribution, homogeneity, individual particle velocities, and overall (e.g., center-of-mass) aerosol velocities (e.g., the number of consumable-aerosol particles of a given size range, per unit volume of air, upon emission of the aerosol toward a consumer); and/or
4) Typical aerosol emission parameters, including the overall flow speed(s) and direction(s) of emitted aerosol and the locations and rates of deposition, relative to the apparatus or consumer (e.g., specific mouth surfaces toward which the aerosol is emitted, on which the aerosol particles are most likely to deposit first).

For any generalized aerosol generation/delivery apparatus, its various design parameters and fluid flow properties contribute to a probability that a certain proportion of the consumable, aerosolizable product is emitted from the apparatus in a way that is useful for the intended use of the apparatus. In general, aerosol flow paths that are:
1) longer (e.g., between an inlet and an outlet);
2) thinner (e.g., have a smaller cross-sectional area);
3) more tortuous (e.g., have a more sinuous path); and/or
4) more encumbered (e.g., more/larger elements like internal partitions, in closer proximity to the flow path);
   generally increase the time it takes for a consumable aerosolizable product to reach a user, and generally increase the likelihood that particles (or a proportion of particles)—subject to such forces as gravity and inertia—are “knocked down” and settle before being emitted from the apparatus. This reduces the proportion of initial consumable product that is ultimately delivered in aerosol form with the desired properties (or decreases the probability that the initial consumable product is ultimately delivered in aerosol form with the desired properties).

Overall, the design of the apparatus, in order for it to be highly useful in the delivery of a consumable, aerosolizable product, is limited by the rates and times it imposes on the passage of aerosolizable product within it. If, for example, the apparatus design implies or requires an “aerosolization time” and/or “aerosol travel time” above a certain critical limit, the aerosol qualities of the product—to the extent it is emitted from the apparatus—may be suboptimal.

These design constraints impose limitations on the initial parameters of the apparatus. Nevertheless, given the relationships among the parameters and their impact on apparatus functionality, significant changes may be made to certain parameters and these can be compensated with changes to other parameters.

In general, embodiments described herein are exemplary of apparatus that appropriately balance the design constraints in such a way as to enable the useful emission of an aerosol product for consumption.

“Breathing” consumable particles is a particularly effective way to enhance, for example, mouth absorption of certain active ingredients.

Molecules—biologically active or not—absorb in the mouth, or elsewhere in the digestive tract, via a three-step process. The first step is dissolution or release from a dosage form, the second is diffusion or convection from the site of dissolution to the absorptive mucosa, and the third is active or passive transport across the mucosa into the bloodstream. As used herein, mucosa (or mucosae in plural form) includes mucous membranes that are linings of mostly endodermal origin, covered in epithelium, which are involved in absorption and secretion. Mucosa lines cavities that are exposed to the external environment and internal organs, and can be contiguous with skin in a person's mouth. For those molecules that absorb across a given mucosal barrier “fast enough” (influenced by hydrophobicity, charge, and molecular size—the more hydrophobic, neutral, and small, the better) the rate and anatomical site of absorption into the bloodstream, once the dosage form is placed in the mouth, is controlled to some degree by the first two steps in the above process, and to some degree by the speed of the digestive process itself—which sweeps ingredients and dosage forms into the gut over some time.

Consumables (e.g., foods, supplements, or drugs) can be delivered as chewable solids, as liquids, as a pill, as a gum, as a strip—or as a fine powder form that gets distributed on the surfaces of the mouth. The dosage form begins to dissolve in the mouth, then is swallowed (possibly to a large extent in the case of, e.g., bread, or a capsule; possibly to a very small extent or not at all in the case of, e.g., a strip or gum). The active ingredient then diffuses to the mucosa.

Depending on the molecule's intimacy with the mucosa, this diffusion may take a significant amount of time; so much so that you tend to swallow quite a bit of the active ingredient before it has the chance to absorb through the mouth. In cases where the diffusion distance to the tongue, for instance, is small, and the likelihood of swallowing is small (e.g., with dissolvable strips), mouth-versus-gut absorption is still influenced by the time of dissolution of the strip, since the longer the strip is dissolving in your mouth, the more times you swallow during the dissolution, and the more likely it is that the dissolved ingredients get swept into the gut.

This is why distributing fine soluble particles (minimally clumped) around the mouth—in particular to the mucosa—is an excellent way/dosage form to enhance absorption in the mouth relative to other dosage forms. The act of “breathing” particles that have the right particle size to minimize or avoid the lungs, using a dispenser with the right device form factor to minimize or avoid the back of the mouth, is a very good and convenient way to do this. This mechanism is important for applications in which it is desirable to avoid gut absorption, and where mouth absorption is viable. This mechanism can also be important for applications where gut absorption of particles and/or other substances carried in and/or dissolved in saliva is desired. The dissolution of substances into the saliva can be enhanced by this delivery method.

In some aspects, a particle delivery apparatus includes an aerosol delivery device for discharge of aerosolized particles to a user.

In some embodiments, the aerosol delivery device includes a mouthpiece defining an inlet, an outlet, and a fluid flow passage extending therebetween, where the inlet is configured to direct the aerosolized particles along the flow passage in a direction generally toward a user's throat.

In some embodiments, the aerosol delivery device includes: a mouthpiece defining an inlet, an outlet, and a fluid flow passage extending therebetween, the inlet configured to direct the aerosolized particles along the flow passage in a direction generally toward a user's throat, and the outlet configured to cause at least a subset of aerosolized particles to agglomerate, self-agglomerate, or de-agglomerate—depending on the nature of the initial aerosolizable product, its particle size distribution, or environmental conditions (e.g., humidity)—in order to improve the emitted aerosol with respect to its intended purpose.

In some embodiments, the aerosol delivery device includes: a mouthpiece defining an inlet, an outlet, and a fluid flow passage extending therebetween, the inlet configured to direct the aerosolized particles along the flow passage in a direction generally toward a user's throat, and the outlet positioned to direct aerosolized particles from the flow passage toward at least one side of a user's mouth.

In some embodiments, the aerosol delivery device includes: a mouthpiece defining an inlet, an outlet, and a fluid flow passage extending therebetween, the inlet configured to direct the aerosolized particles along the flow passage in a direction generally toward a user's throat, and the outlet positioned to direct aerosolized particles from the flow passage toward one or more specific surfaces or materials within a user's mouth (e.g., inner cheek, sublingual area, top surface of tongue, side of tongue, teeth, plaque, gums, saliva).

In some embodiments, the aerosol delivery device includes a mouthpiece defining an inlet, an outlet, and a fluid flow passage extending therebetween, the inlet configured to direct the aerosolized particles along the flow passage in a direction generally toward a user's throat; and a deflector disposed along the flow passage, the deflector configured to redirect at least a portion of the aerosolized particles along the flow passage from the inlet toward the outlet, the outlet positioned to direct aerosolized particles from the flow passage toward at least one side of a user's mouth (e.g., top, bottom, left, right; palate; on tongue or below tongue; inner cheek; etc).

Embodiments can include one or more of the following features.

In some embodiments, the deflector comprises a curved portion. In some cases, the curved portion is configured to redirect at least a portion of the aerosolized particles by about 90 degrees. In some cases, the curved portion comprises a substantially continuous curve extending from the inlet to the outlet.

In some embodiments, the deflector is configured to redirect aerosolized particles about 90 degrees along the flow passage.

In some embodiments, deflection is not achieved by the presence of a deflector that is an integral element of the apparatus. For example, deflection can be achieved—to an extent sufficient for the intended purpose of the overall delivery system—by elements external to the apparatus, or not part of the original design of the apparatus. Such deflectors include physiological members (e.g., the tongue, teeth, gums, inner cheek, fingers, etc. of the user).

In some embodiments, the mouthpiece further defines an inhaler orifice in fluid communication with the fluid flow passage, the inhaler orifice positioned to direct at least a portion of inhaled fluid toward the user's throat. In some cases, the inlet defines a first plane, the outlet defines a second plane, the inhaler orifice defines a third plane, and at least one of the first and third plane is nonparallel to the second plane. At least one of the first and third plane can be substantially perpendicular to the second plane. The first plane can be substantially parallel to the third plane.

In some embodiments, the pressure drop of fluid flowing between the inlet and the inhaler orifice is greater than the pressure drop of fluid flowing between the inlet and the outlet.

In some embodiments, the minimum cross-sectional open area of the flow passage between the inlet and the inhaler orifice is less than the minimum cross-sectional open area of the flow passage between the inlet and the outlet.

In some embodiments, the cross-sectional open area of the inhaler orifice is less than the cross-sectional open area of the outlet. In some cases, the ratio of the cross-sectional open area of the inhaler orifice to the cross-sectional open area of the outlet is between about 0.1 to about 0.9.

In some embodiments, the outlet comprises a plurality of orifices defined by the mouthpiece, the plurality of orifices disposed between the inlet and the inhaler orifice. In some cases, at least some of the plurality of orifices are spaced along an axis substantially parallel to an axis defined by the inlet and the inhaler orifice. In some cases, at least some of the plurality of orifices are spaced about a circumference of the mouthpiece.

In some embodiments, the flow passage is elongate in a direction between the inlet and the inhaler orifice. In some cases, the outlet comprises a plurality of orifices axially spaced from one another along an axis substantially parallel to an axis defined by the inlet and the inhaler orifice.

In some embodiments, the deflector is movable to restrict the flow of fluid through the inhaler orifice. In some cases, the deflector is movable by fluid flowing through the flow passage. In some cases, the deflector comprises a pinwheel disposed along the flow passage, the pinwheel rotatable about an axis substantially parallel or substantially perpendicular to an axis defined by the inlet and the inhaler orifice. In some cases, the device also includes a track disposed substantially within the mouthpiece, along the flow passage, wherein the deflector is configured to move along the track. For example, at least a portion of the track can extend in a direction parallel to an axis defined by the inlet and the inhaler orifice.

In some cases, the device includes a track disposed substantially within the mouthpiece, along the flow passage, wherein a movable element (e.g., a small sphere) is configured to move along the track. For example, at least a portion of the track can extend in a direction parallel to an axis defined by the inlet and the inhaler orifice. In some cases, the movable element can block or give access to fluid flow passages, thus controlling flow parameters and pressure drops. These flow modifications can, in turn, cause air or aerosol to substantially flow along a certain path (or paths), or out of a particular inhaler outlet or orifice (or outlets or orifices).

In some embodiments, the deflector and the mouthpiece define a substantially helical flow path along at least a portion of the flow passage between the inlet and the inhaler orifice. In some cases, the outlet is defined along a portion of the mouthpiece defining the helical flow path.

In some embodiments, the outlet(s), deflector(s), and/or orifice(s) (where present), cause the aerosolizable product to agglomerate or de-agglomerate. For example a deflector, by virtue of the airflow pathway it causes an aerosol to follow, may subject larger aerosol particles, or aerosol particles consisting of multiple constituents, to forces that cause smaller sections or constituents to separate from each other, resulting in finer particles. As another example, a deflector may subject smaller aerosol particles to forces that bring them together and cause them to physically (or chemically) combine into, or onto, larger particles, or combine/dissolve within a medium (e.g., an oil). Overall, particles emitted from the apparatus, by virtue of the design of the apparatus, may be physically (or chemically) different from the particles initially introduced into the apparatus. In particular, the particle size distribution can be thus changed, resulting in an aerosol with more desirable properties for a given intended purpose.

In some aspects, a particle delivery apparatus includes: an aerosol delivery device for discharge of aerosolized particles to a user. The aerosol delivery device includes: a mouthpiece defining an inlet, an inhaler orifice, a fluid flow passage extending therebetween, and an outlet in fluid communication with the flow passage, the inlet configured to direct fluid along the flow passage in a direction generally toward a user's throat; and a first reservoir defining a first outlet directed generally toward at least one side of a user's mouth, the first reservoir configured to support aerosolized particles, and the first reservoir in fluid communication with the flow passage such that fluid flowing through the flow passage displaces at least a portion of the aerosolized particles through the first outlet. Embodiments can include one or more of the following features.

In some embodiments, the reservoir is releasably coupled to the mouthpiece. In some cases, the reservoir is disposable.

In some embodiments, the pressure drop of fluid flowing through the first reservoir is less than the pressure drop of fluid flowing through the flow passage.

In some embodiments, the outlet comprises a plurality of orifices.

In some embodiments, devices also include a second reservoir defining a second outlet directed generally toward at least one side of a user's mouth, the second reservoir configured to support aerosolized particles, and the second reservoir in fluid communication with the flow passage such that fluid flowing through the flow passage displaces at least a portion of the aerosolized particles through the second outlet. In some cases, the first outlet and the second outlet define an axis substantially perpendicular to an axis defined by the inlet and the inhaler orifice.

In some aspects, a method of delivering food includes: receiving aerosolized particles in a mouthpiece defining an inlet, an outlet, and a fluid flow passage extending therebetween; directing the aerosolized particles along the flow passage from the inlet in a direction generally toward a user's throat; redirecting at least a portion of the aerosolized particles from the direction generally toward a user's throat generally toward the outlet, the outlet positioned to direct aerosolized particles from the flow passage toward at least one side of a user's mouth.

Embodiments can include one or more of the following features.

In some embodiments, methods also include directing inhaled fluid toward an inhaler orifice defined by the mouthpiece, the inhaler orifice positioned to direct at least a portion of inhaled fluid toward the user's throat. In some cases, redirecting at least a portion of the aerosolized particles comprises restricting flow to the inhaler orifice.

In some embodiments, redirecting at least a portion of the aerosolized particles comprises moving a deflector disposed in the flow passage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

The advantages of the disclosure described below, as well as further advantages of the disclosure, can be better understood by reference to the description taken in conjunction with the accompanying figures, in which:

FIGS. 1A and 1B are schematics of an embodiment of a particle delivery apparatus, respectively, before use and during use.

FIGS. 2A and 2B are perspective views of a particle delivery device.

FIGS. 2C and 2D are, respectively, an exploded perspective view and a cut-away perspective view of the particle delivery device of FIGS. 2A and 2B.

FIG. 2E is a cut-away perspective view of the particle delivery device of FIGS. 2A and 2B.

FIGS. 2F and 2G, respectively are a cross-sectional views of the particle delivery device of FIGS. 2A and 2B and of a portion of the particle delivery device of FIGS. 2A and 2B.

FIG. 3 is a schematic of a particular embodiment of the particle delivery apparatus and a diagram for its use and operation.

FIG. 4 presents multiple views of an exemplary embodiment of a mouthpiece 112.

FIG. 5 presents multiple views of an exemplary embodiment of an end cap 114.

FIG. 6 presents multiple views of an exemplary embodiment of a capsule 116

FIG. 7 is a graph from a HELOS-RODOS particle size analysis of dried, crushed, and sieved mint leaves.

FIGS. 8 and 9 are photographs of a particle delivery device. The particle delivery devices include a housing, a mouthpiece formed therewith, an airflow directing element attached therewith via bridges, a capsule having air passageways and grating, and a cap having air passageways and capable of snapping together with both the capsule and the housing. In some embodiments, the grating, here part of the capsule, serves as an aerosol-generating device.

FIG. 10 sets forth the specifications of a particular embodiment of a particle delivery apparatus. The particle delivery apparatus includes a housing, a mouthpiece formed therewith, an airflow directing element attached therewith via bridges, a capsule having air passageways and grating, and a cap having air passageways and capable of snapping together with both the capsule and the housing.

FIGS. 11A and 11B are perspective views of an embodiment of a particle delivery apparatus, respectively, in closed and open positions.

FIGS. 11C-11I are detail views of different portions of the embodiment of the particle delivery apparatus shown in FIGS. 11A and 11B.

FIGS. 12-18 are schematics of particle delivery devices.

FIGS. 19A-19D are perspective views of a particle delivery device.

### DETAILED DESCRIPTION

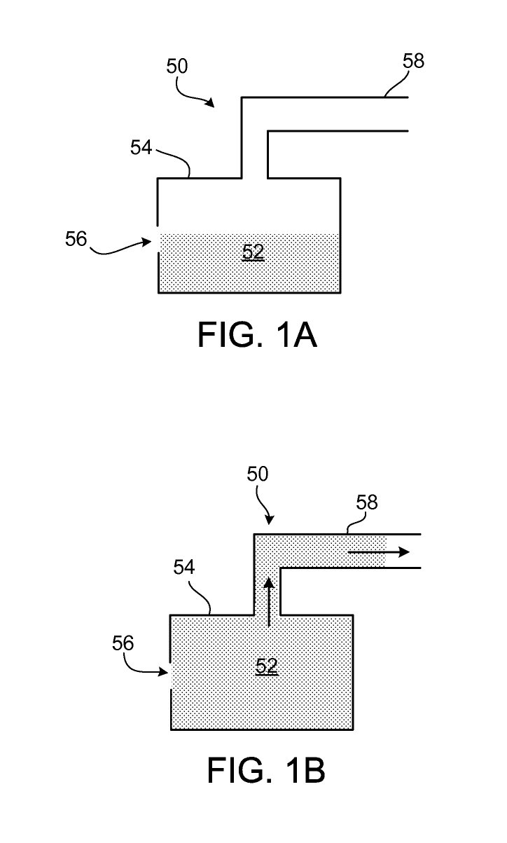

Our approach is based, at least in part, on the realization of a new form of consumable product and methods and apparatus for the delivery thereof. More specifically, the delivery technology and approach is directed to aerosolized particles (e.g., food products) and a particle delivery method and apparatus designed to generate and deliver such products to a subject. Such devices can deliver food substances into the mouth by aerosol wherein the aerosol cloud is generated and delivered to the mouth through a natural inspiration or expiration maneuver and wherein the design of the mouthpiece of the device is such that the airborne particles (e.g., food particles) are diverted away from the back of the throat to limit entry into the respiratory system. Referring to FIGS. 1A and 1B, a particle delivery apparatus 50 includes an aerosol generating device, in which inhalation triggers the aerosolization of a food product 52 and subsequent delivery of the aerosolized food product to the mouth of a subject. The particle delivery apparatus 50 includes a compartment 54 containing the food product 52 (e.g., a powdered food). The compartment 54 has an air intake passage 56 and is connected to a mouthpiece 58. The air intake passage 56, the compartment 54, and the mouthpiece 58 allow for the passage of air such that airflow generated by inhalation aerosolizes the food product 52 and transports the aerosolized food product out of the compartment 54, through the mouthpiece 58 and into the consumer's mouth. Although described with respect to delivery of food products, the devices and methods discussed can be used for generation and delivery of other products (e.g., medicinal products).

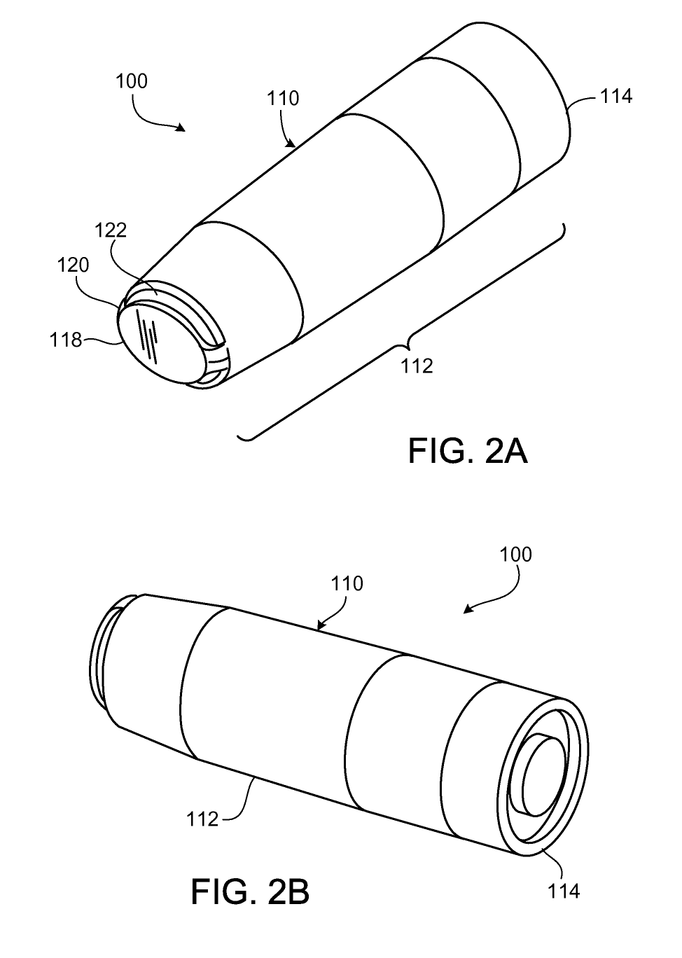

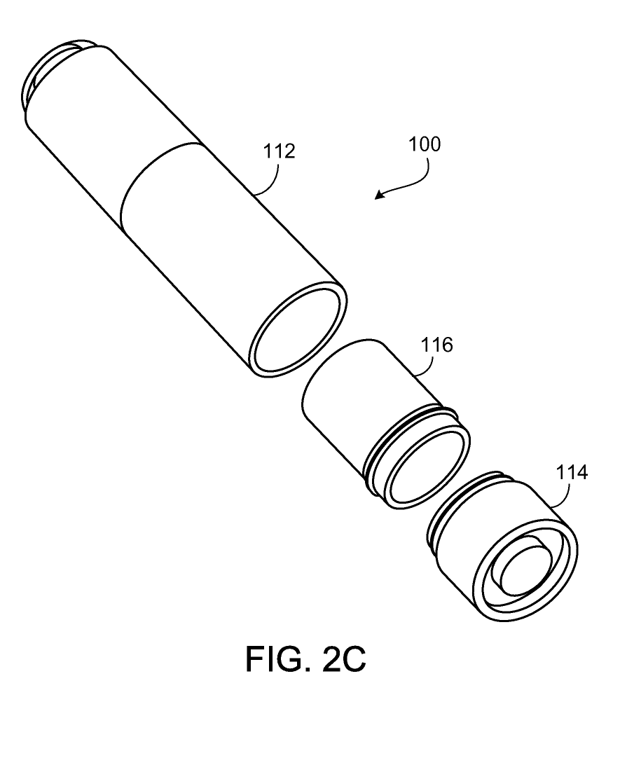

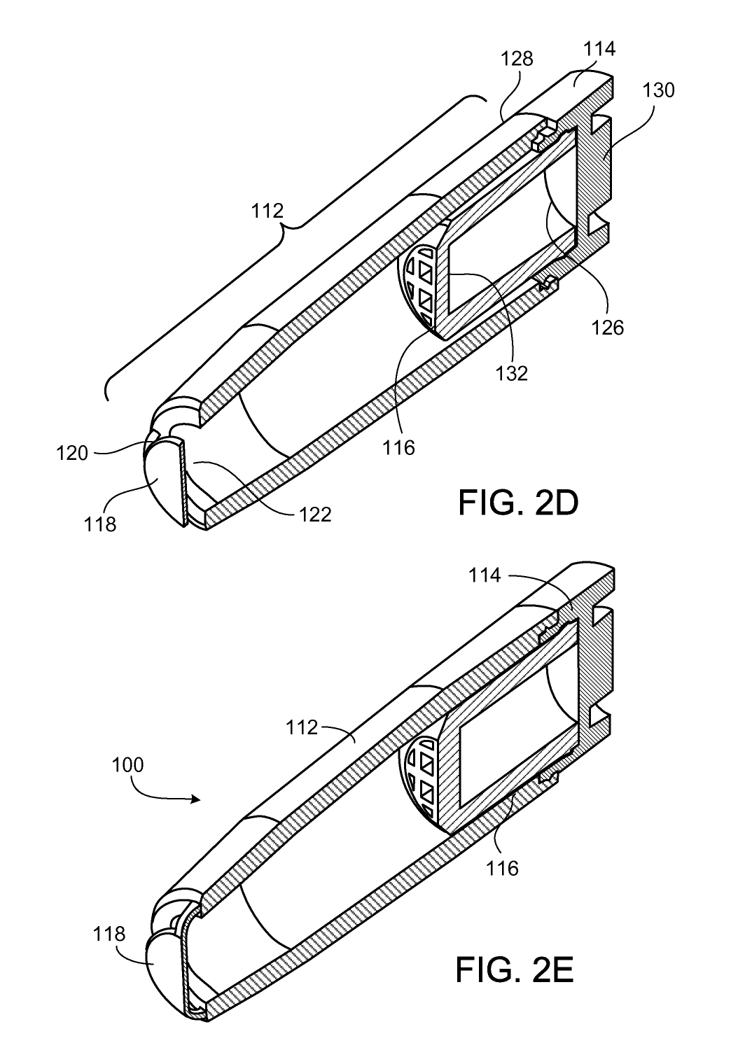

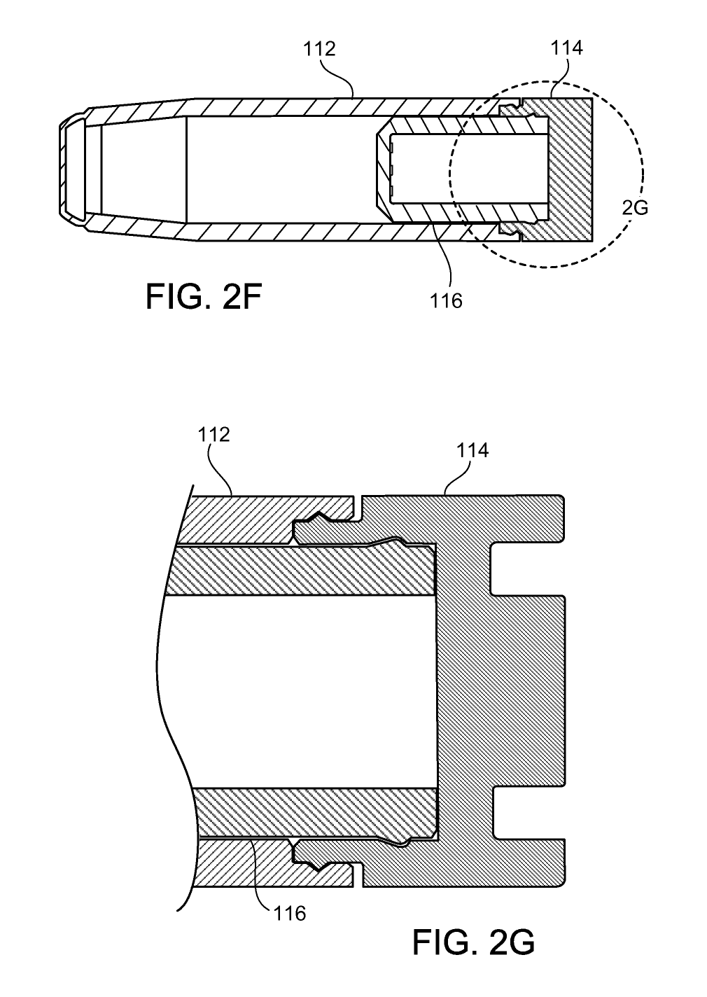

Referring to FIGS. 2A-2F, a particle delivery device 100 includes a housing 110 with a mouthpiece 112 and a detachable end cap 114. The particle delivery device 100 is sized such that a user can easily hold the device in one hand while using the device 100 to generate and deliver an aerosolized food product. An airflow directing or deflection member 118 is disposed at one end of the mouthpiece 112 with bridges 120. The bridges 120 position the airflow directing member 118 in a location spaced apart from a plane of an outlet 122 of the mouthpiece 112. The end cap 114 is attached to the end of the mouthpiece 112 opposite the airflow directing member 118.

As can be seen in FIG. 2D, the mouthpiece 112 defines a fluid flow passage extending from an inlet 124 to the outlet 122 of the mouthpiece 112. The end cap 114 has air passageways 126 extending from one face of the end cap 114 to an opposite face of the end cap 114. When the end cap 114 is attached to the mouthpiece 112 on the inlet end of the mouthpiece 112, the mouthpiece 112 and the end cap 114 together define a flow path through the housing 110. Thus, when a user places the outlet 122 of the mouthpiece 112 in his or her mouth and inhales, air flows through end cap 114, into the inlet 124 of the mouthpiece 112, and through the mouthpiece 112 to the outlet 122 of the mouthpiece 112. Contact with the airflow directing member 118 deflects the air flowing out of the mouthpiece 112.

In some embodiments, the airflow-directing element and/or deflection member may take any of a variety of forms (not necessarily that of a disc), in order to divert the airflow exiting the mouthpiece and entering the mouth, away from a straight trajectory toward the throat and lungs. For example, there may be one or more openings near the top of a mouthpiece, which may be offset relative to each other, at different heights, of different sizes, of different areas, etc., which maintain the general blockage of the direct linear path toward the back of the mouth, and generally divert the airflow and aerosol such that it goes in more lateral directions.

In some embodiments, the airflow-directing element is a thin disc with a flat surface generally perpendicular to the axis of the mouthpiece and in opposition to the general airflow direction in the mouthpiece. In some cases, the disc may be mounted to the mouthpiece via one or more “bridges”, which may, for example, hold the disc slightly above, below, or at the same level as the edge of the mouthpiece, allowing air, and the aerosolized food product to pass around the disc. In various embodiments, the disc may have a diameter smaller, equal to, or larger than the opening of the mouthpiece. Additionally, the disc may be of any desired shape, for example, an elliptical shape or round shape. The airflow-directing element redirects the aerosol to the sides of the mouth (e.g. top, bottom, left, right surfaces within the mouth), thereby limiting flow of the aerosol toward the throat where it might elicit a coughing reflex. Instead, the aerosolized food product deposits on the tongue or other parts of the mouth where it can be sensed and appreciated rather than carried deeper into the respiratory tract. In some embodiments, the airflow-directing element is of a different shape, size, and/or design but similarly serves to redirect the aerosolized food product so as to limit the coughing reflex and/or to enhance the taste experience. Testing of a variety of disc sizes and positions has shown that these two parameters can impact likelihood of coughing. For example, it was found in preliminary tests that a disc whose diameter is roughly equal to that of the external diameter of the mouthpiece, and that is placed close to the mouthpiece, is generally more effective in redirecting the aerosol and limiting coughing, than one whose diameter is roughly equal to that of the internal diameter of the mouthpiece (thus smaller) and that is placed at a greater distance from the mouthpiece (leaving a larger space for the aerosol to pass through).

In this embodiment, the end cap 114 is formed of a resilient material. A first end 128 of the end cap 114 has an outer surface that is sized and configured to provide a snap-fit engagement with the inner surface of the corresponding end of the mouthpiece 112. In some embodiments, other forms of engagement are used instead of or in addition to snap-fit engagement to attach the end cap 114 to the mouthpiece 112. For example, in some embodiments, the end cap 114 and the mouthpiece 112 have threads and are screwed together.

The mouthpiece 112 together with the end cap 114 (i.e., the housing) define an interior cavity sized to receive a capsule 116 such as, for example, a capsule 116 containing a powdered food product (not shown). The capsule 116 is configured to provide, or otherwise be in, fluid communication between the contents of the capsule 116, for example, a powdered food product, and the mouthpiece. In this embodiment, the capsule 116 has an open end 130 and an opposite aerosol generating end 132. The open end 130 of the capsule 116 fits within the first end 128 of the end cap 114 and is sized and configured to provide a snap-fit engagement with the inner surface of the first end 128 of the end cap 114. In some embodiments, the capsule may be snapped or screwed into the housing. In some embodiments, the capsule includes an open end that may be covered (in certain embodiments, only at certain times) by the cap, for example, by snapping or screwing. In some embodiments, the inlet end of the capsule defines air passages rather being open.

Referring to FIG. 2F, in some embodiments, the capsule 116 snaps into the cap 114 by a full annular snap mechanism on the inside of the cap 114, and the cap 114 snaps into the mouthpiece 112 by an interrupted snap mechanism. The device may thus be designed so that it is typically more difficult to separate the cap 114 from the capsule 116 than to separate the cap 114 and/or capsule 116 from the mouthpiece 112. A user can then easily replace the capsule 116 and/or cap 114 by removing it from the mouthpiece 112, with minimal risk of accidentally detaching the capsule 116 from the cap 114.

Some embodiments may be further enhanced by incorporating snap mechanisms that facilitate the use and functionality of the device. For example, a device may incorporate snap mechanisms to facilitate the use of a mechanism like the one described above that allows for the opening and closing of air passageways. For example, the mouthpiece and capsule can be designed such that they are able to connect by one (or more) snap mechanism(s), and the capsule and cap are able to connect by two (or more) snap mechanism(s). For example, the mouthpiece may be connected to the capsule by one relatively weak snap interface, and the capsule may be connected to the cap by two relatively strong snap interfaces. In some embodiments, these snap mechanisms can: (1) hold the capsule (or, more generally, hold one end of the food-containing apparatus) to the mouthpiece (or, more generally, to the delivery apparatus) (“snap A”); (2) hold the capsule and cap (or, more generally, hold together the components of the food-containing apparatus) in an initial “closed” configuration that minimizes powder loss (especially relevant during shipping, handling, etc.), and may also serve to provide a protected, airtight or nearly airtight environment for the preservation of the food product before use (“snap B”); and (3) after user intervention, reconnect the capsule and cap (or, more generally, the components of the food-containing apparatus) to maintain a new “open” configuration in which air can flow through the apparatus and enable subsequent aerosolization of the food product (“snap C”).

The forces required to actuate each of these snaps plays a role in the functionality and ease of use of the device. They may be configured to allow use as follows: (1) The user attaches the capsule/cap component to the mouthpiece. Snap A is actuated. Now, the capsule is hidden within the mouthpiece and the cap. (2) The user now pulls back on the cap, undoing Snap B. With a strong Snap A, the capsule stays connected to the mouthpiece and the cap slides away from the mouthpiece. This relative motion between the capsule and cap allows for the air passageways to open, as described earlier. (3) The user continues to pull the cap back until Snap C is actuated, locking the capsule and cap in place in such a way as to leave the air passageways open. The user can now inhale and have the food product aerosolize and be delivered to the mouth. Once the food product is consumed, the high strength of this snap (C) allows the user to pull out the capsule/cap from the mouthpiece and replace it with a new capsule/cap, with minimal risk of separating the capsule from the cap instead (the capsule is simultaneously connected to the mouthpiece via snap A and connected to the cap by snap C; since snap C is engineered to be stronger than snap A, a force applied by the user that pulls the mouthpiece and cap in opposite directions generally leads to the capsule and cap detaching from the mouthpiece as one unit, thus undoing snap A). In some embodiments, snap C is also important in that it minimizes the user's ability to completely separate the capsule and cap, even after the mouthpiece is removed. In some cases, it may be desirable to prevent a user from attempting to add his/her own product, or otherwise tamper with the food product or food-containing compartment.

In many instances, variations of some embodiments may be designed without, in many instances, affecting the function of the overall device. For example, the cylindrical nature of the device may be modified, for example, for aesthetic effect, as may the overall length of the device. Alternatively, or in addition, the aerosol generating device, for example, the airflow disrupting element such as a grating, may be incorporated into the cylindrical mouthpiece unit. In some embodiments, the aerosol generating device may include more than one component. For example, a grating and/or the airflow passageways in the cap may play individual roles in generating turbulence that leads to aerosolization, or both may be needed. In general, there may also be multiple configurations of gratings, airflow passageways, dimensions etc, to produce the right aerosolization airflow.

In some embodiments, the dimensions of the device may be selected so that, while preserving the appropriate airflow dynamics, standard medical capsules may be used directly as the compartment, or may to some extent replace the previously described capsule and/or cap, or in another way simplify the process of loading, storing, and releasing the powder.

In some embodiments, the capsule and/or cap have concave inner spaces, and, after powder is filled into either or both of them, the two units snap or screw together to form a largely closed interior chamber. The capsule, or another component of the device, should further include an aerosol generating device, for example, an airflow-disrupting “grating”, through which air and powder flow, thereby yielding an aerosol for delivery to the user. The cap and/or the capsule should typically include air passageways, for example, on the respective ends of the enclosed compartments, so as to allow air to flow through upon inhalation. The design, for example, the size or shape of the air passageways, should provide sufficient airflow while minimizing powder loss.

In some embodiments, the cap 114 and/or the capsule 116 is designed to minimize powder loss. For example, as shown in FIG. 1E, the air passageways angle out to the sides, rather than straight through to the bottom, so as limit powder from falling out due to gravity, even when the device is held upright. When the powder is inside the capsule/cap, and shaking or other movements are minimal, powder may accumulate against the bottom surface of the passageways but minimally fall out through the side passageways.

In some embodiments, the need for balance between airflow and minimal powder loss may be achieved by a mechanism that enables air passageways to be alternatively open or closed. For example, in some embodiments, the capsule and cap components may fit together but remain capable of sliding against each other, to enable two configurations: in the closed configuration, the two are closer together, with elements at the base of the capsule blocking the air passageways of the cap; in the open configuration, the capsule and cap are separated slightly, allowing air to flow through the air passageways in the cap.

In some embodiments, the mouthpiece, capsule, and/or cap are designed for single use (perhaps disposable) or, alternatively, designed for multiple use. For example, in some embodiments, the capsule and cap may be disposable, and, optionally, available with a variety of food powders, while the mouthpiece may be reusable. In certain embodiments, pre-filled standard-sized capsules, for example, a gel capsule or blister pack, can be used. Such embodiments allow for easier filling, substitution, cleaning, and disposal. In addition, such embodiments allow for manufacture of multiple dose capsules. Such pre-filled capsules could be punctured, torn, cut or broken by design elements within the housing (for example, sharp points, blades, compressing the device, or twisting the device etc.) prior to use. The food product may thus be released into a chamber, for example, and become more susceptible to airflows generated during inhalation or activation; or the food product, as another example, may remain substantially within the original container but now be in fluidic communication with, and thus now susceptible to, airflows generated during inhalation and/or activation; etc. After activation and use, the emptied capsule could be removed from the compartment and disposed of conveniently. Alternatively, the capsule can be designed for multiple uses. For example, the capsule may be refillable.

In some embodiments, the housing is designed to allow for the incorporation of at least 2, for example, 3, 4, 5, 6, 7, 8, 9 or 10, capsules, thereby allowing, for example, the user to mix and match a variety of flavors in various amounts as desired. In some embodiments, the housing could be designed to allow for the loading of a set of multiple capsules to be activated one at a time, thus reducing the frequency of removing and replacing spent capsules.

In some embodiments, the device is designed for use by at least 2, for example, 3, 4, 5, 6, 7, 8, 9 or 10, users. For example, the device could be designed with multiple branches, each designed with an airflow directing element, so as to allow for simultaneous use by multiple users.

In certain aspects, the device includes a housing, a capsule and a cap. In alternative aspects, a device includes the housing and a cap, wherein both the housing and the cap are designed for use with capsules, for example, disposable or refillable capsules. In other aspects, the device encompasses disposable or refillable capsules. In other aspects, the device encompasses mouthpieces, used with a variety of aerosolized food products, aerosolized food product sources, and/or aerosolized food product containers.

It should be noted that the functionalities (i.e., food product containment, aerosol generation, aerosol delivery, airflow (and aerosol) direction, etc.) of the mouthpiece, capsule, cap, grating, mouthpiece disc, etc. may, in some embodiments, be associated with one or more potentially different physical units, while maintaining the same overall functionality. For example, in some embodiments, a single device unit may incorporate all functional aspects. In some embodiments, a mouthpiece may contain an aerosol generating device, an aerosol delivery device, and an airflow- (and aerosol-) directing device, and the food product container may be separate. In some embodiments, as previously described, food product may be contained within a capsule and cap, an aerosol generating device may be part of a capsule, and a mouthpiece with airflow-directing elements may be used to deliver the aerosol from the capsule/cap to the user.

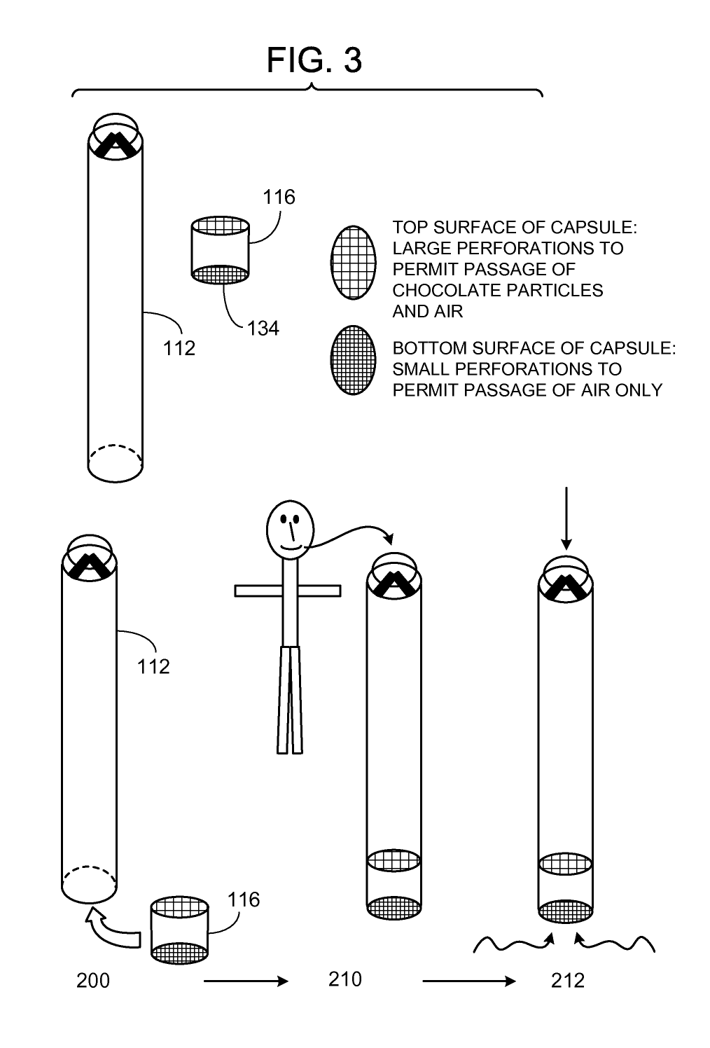

Referring to FIG. 3, a user operates a particle delivery device 100 by loading the device 100 (step 200); bringing the device 100 to the user's mouth (step 210); and inhaling through the mouthpiece 112 (step 212) thereby causing air to enter the cap and the capsule through the air passageways. The air compels the food powder present in the capsule 116 to aerosolize through the aerosol generating device, for example, the grating, and subsequently enter the user's mouth via the mouthpiece 112.

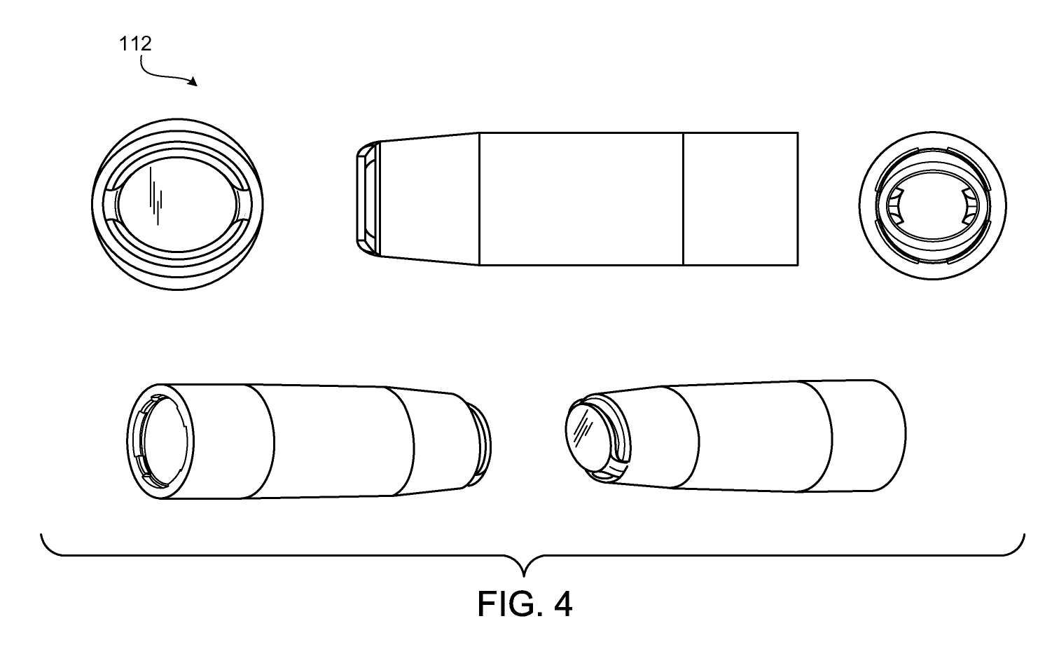

FIG. 4 presents multiple views of an exemplary embodiment of a mouthpiece 112.

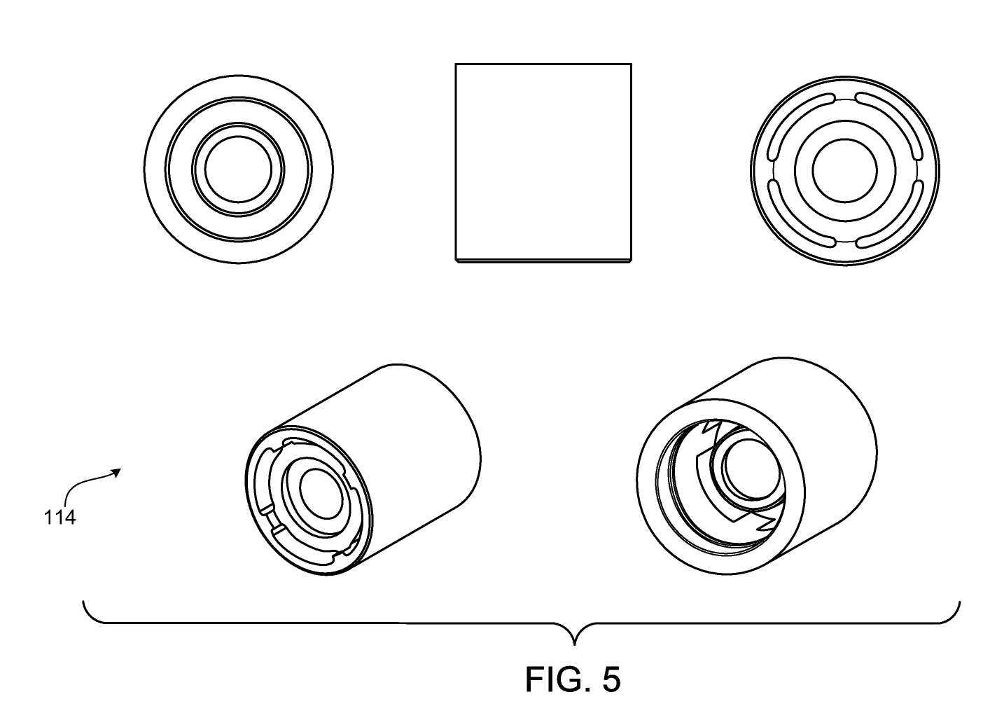

FIG. 5 presents multiple views of an exemplary embodiment of an end cap 114.

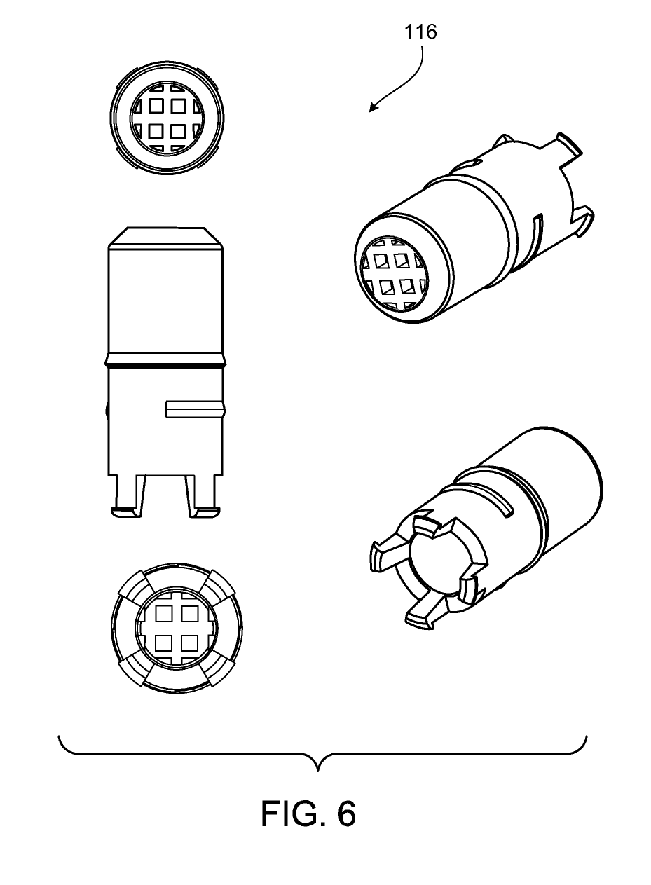

FIG. 6 presents multiple views of an exemplary embodiment of a capsule 116.

In some of the embodiments described above, the aerosol is generated at a particular point in time or over a small interval of time corresponding to a specific activation step, and/or the aerosol is generated by a user-dependent step. For example, in some cases aerosol generation is associated with one or more inhalation maneuvers by the user. In many of these embodiments, the food product is in a solid state, and may be a substantially dry powder.

### Activation of Aerosolization and Delivery of Food Product

The aerosol generating device is any device capable of producing an aerosol of desired characteristics (i.e., particle size, airborne time/suspension duration, emitted dose, etc.). In addition to the aerosol generating device, there may be a delivery device, such as an additional airflow constraining device, a confined space in which the aerosol is contained, an air passage in an inhaler, a mouthpiece, airflow-directing elements, or other devices or structures, that enable, facilitate, or optimize the delivery of the aerosol to the subject's mouth. For example, FIGS. 2A-6 illustrate the capsule and cap, which in many embodiments serve as a food product container and incorporate an aerosol-generating device (consisting primarily of the grating). In many embodiments, the capsule and cap are connected to each other and to a mouthpiece with airflow-directing elements, where the mouthpiece would serve as a delivery device.

By controlling gravitational and inertial forces, the airflow-directing elements found in some embodiments enable delivery of the aerosol cloud substantially to surfaces within the mouth rather than further down the respiratory tract. This aspect of the technology is highly relevant to a number of potential applications of food aerosols. Indeed the same such delivery device can make possible delivery of a wide range of food aerosols, generated in a number of different ways, to a consumer, while minimizing or eliminating coughing and potential interactions with surfaces of the respiratory system beyond the mouth.

The design of any of the devices or structures associated with this technology may also take into consideration and attempt to reduce any tendency to cough, gag, or otherwise react unfavorably to the aerosol.

These devices, and associated devices (such as a food-containing device), can be embodied in a vast number of different ways. The devices described herein are meant to be exemplary.

Triggering the aerosolization of the food product and subsequent delivery of the resulting aerosolized food product may occur by a variety of means including, but not limited to, acts of respiration, device activation, bodily displacement, aerosol displacement and a combination thereof. For example, such acts may include:
a) an act of respiration, for example, by inhalation on a mouthpiece, resulting in exposure of the food product to the aerosol generating device and delivery of the aerosolized food product to the mouth; and/or
b) an act of device activation, including, but not limited to, the activation of an ultrasound source, the actuation of a pump, the activation of a compressed air source, the activation of an impeller, the puncturing of a container, the opening of an air passage, that at least in part causes or helps to cause a food product to aerosolize (the aerosol thus formed may be in a substantially confined space (e.g., a spacer), or a substantially open space (e.g., as a “cloud” in air or in a confined structure)); and/or
c) an act of respiration directed “on” or “toward” an aerosol (e.g., that is contained in a spacer device, freely floating as a cloud or contained within a larger structure), and that may be facilitated by the use of a straw, mouthpiece, or other apparatus, thereby leading to food deposition substantially in the mouth; and/or
d) an act of bodily displacement, such as walking or leaning (possibly in conjunction with a particular placement or positioning of the mouth, tongue, or other body part in a specific way), that exposes a subject's mouth to an aerosol cloud, or portion thereof, thereby leading to food deposition substantially in the mouth, and/or
e) an act of aerosol displacement, caused by, for example, an air current, a thermal or pressure gradient, inertial impaction, diffusion, or gravity, that brings an aerosol cloud, or portion thereof, to a position so as to expose a subject's mouth to the aerosol cloud, thereby leading to food deposition substantially in the mouth (even where aerosol displacement results in dilution of the particle concentration and spreading out the cloud); and/or
f) an additional act of device activation, device use, space constraining, airflow confinement, etc., or of placement or positioning of the mouth, lips, tongue, jaw, head, or other body part in a particular configuration, shape, etc.; or other additional action that helps produce the proper aerosolization and/or delivery and/or tasting of the food product (e.g., use of a food straw, opening/closing of a containing chamber, lifting of the tongue to divert airflow, etc.). Such acts may be used to help reduce a tendency to cough, gag, or otherwise react unfavorably to the food product.

All references to a powder, liquid, aerosol, cloud, particle, etc. made herein may equivalently refer to some fraction or portion of the total amount of the powder, liquid, aerosol, cloud, etc.

The device itself may be designed for single use (for example, disposable) or multiuse, for example, where the dosage capsule is replaced or the dosage chamber refilled. Alternatively, or in addition, parts of the device, for example, the mouthpiece, the food-containing apparatus, the capsule, and/or the cap, may be disposable. In some embodiments, the device may incorporate a force-generating mechanism, such as a pump or compressed air source, to aerosolize the food product. In some embodiments, the device may incorporate a propellant.

In some embodiments, the device may be designed for “single action”, “repeated action”, or “continuous action” aerosolization and/or delivery, depending on whether it is intended to aerosolize and/or deliver the product in a single, short-term step (e.g., one inhalation on an inhalation-triggered apparatus), in multiple discrete steps (e.g., multiple inhalations on an inhalation-triggered apparatus), or over a longer-term continuous step (e.g., maintaining an aerosol cloud in open air), where “step” can refer to any combination of simultaneous and/or sequential processes by which the device aerosolizes and/or delivers the product. Many factors, including whether the device is intended for use by one subject or multiple subjects at a time, will help determine which of these step sequences (if any) is appropriate for any particular embodiment.

In some embodiments, an aerosol is generated by an expiratory breathing maneuver, in which air emitted by a user either directly or indirectly causes a consumable product to aerosolize.

The device might also include additions, such as spacers, lights, valves, etc., to enhance the visual effect and/or the control over the aerosol and/or dosage. These additions may also enhance the experience of inhaling the aerosols.

In some embodiments, a user places his/her tongue near an outlet (or inhaler orifice) in order to alter the speed and/or direction of the aerosol emitted from the apparatus. In some cases, the user may position the outlet such that the aerosol is emitted toward the sublingual area. In some cases, the user may position the outlet such that the aerosol is emitted toward to lower side of the tongue, with the tongue in an elevated position (i.e. with the tip of the tongue generally closer to the top of the mouth than a region of the tongue closer to the throat). In some cases, an inspiratory or sipping maneuver under such conditions will cause aerosol particles to enter the mouth and divert to a desired surface or material within the mouth (e.g., the sides of the mouth, the top of the tongue, saliva, taste buds). In some cases, such conditions will limit undesirable side effects, such as coughing.

In some embodiments, a user places his/her teeth near an outlet (or inhaler orifice) in order to alter the speed and/or direction of the aerosol emitted from the apparatus. In some cases, under such conditions, aerosol particles with hygienic, “freshening”, or other qualities are thus diverted toward surfaces where these particles can be most beneficial (e.g., gum surfaces).

In some embodiments, other physiological members are used to favorably alter the speed and/or direction of the aerosol emitted from the apparatus.

In some embodiments, the body of the entire apparatus, or parts of the apparatus, could be manufactured of an edible/ingestible substance, such as a cookie, cracker, chocolate, or sugar product, etc. This would allow the device to be enjoyed either during the aerosol delivery or afterwards, thus enhancing the overall experience.

In some embodiments, the device may be similar to an inhaler or inhalation device, such as a dry powder inhaler (DPI) or metered dose inhaler (MDI); a “pot” that holds an ultrasound source and confines somewhat the aerosol cloud produced by the source; a “fountain” that ejects and/or circulates the aerosol; a hand-held pump device; a compressed air device; a food straw device; a multi-person, communal device; a tabletop device. A variety of materials may be used to form the device, or parts thereof, including: plastics (e.g. polycarbonates, which are relatively strong, polypropylene, acrylonitrile butadiene styrene, polyethylene, etc.), various metals, glass, cardboard, rigid paper, etc.

In some embodiments, the aerosolized food product should be of a determined size, i.e., of sufficient size to limit entry into the respiratory tract but of small enough size to allow for suspension in the air. In some embodiments, particle size may be a manufacturing requirement of pre-atomized, generally solid food products, for example the food products placed inside the capsule/cap of certain embodiments, or certain dry food products used in association with an air pump or compressed air source. In some embodiments, particle size may be a requirement of the aerosol-generating device, for (generally liquid) food products that are only atomized upon aerosol generation, for example the food products used in association with ultrasound sources to produce an aerosol cloud.

In some embodiments, the predetermined, mean size of the aerosolized food product is at least 1, 2, 3, 4, 5, 10, 15, 20, 25, 30, 35, 40, 45, 50, 55, 60, 70, 75, 80, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 325, 350, 375, 400, 425, 450, 475, or 500 microns. In some embodiments, the predetermined, mean size of the aerosolized food product is less than 500, 450, 400, 350, 325, 300, 275, 250, 245, 240, 235, 230, 225, 220, 215, 210, 205, 200, 195, 190, 185, 180, 175, 150, 140, 130, 120, 110, 100, 90, 80, 70, 60, 50, 40, 30, 20, or 10 microns in size. Ranges intermediate to those recited above, e.g., about 50 microns to about 215 microns, are also intended to be part of this disclsoure. For example, ranges of values using a combination of any of the above recited values as upper and/or lower limits are intended to be included.

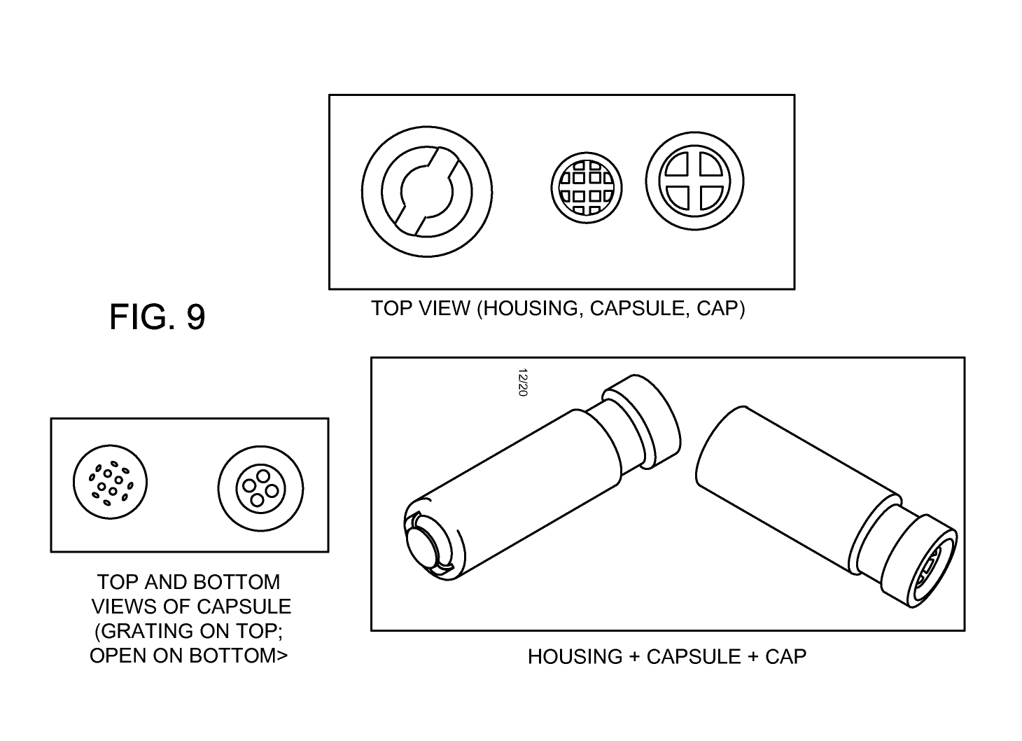

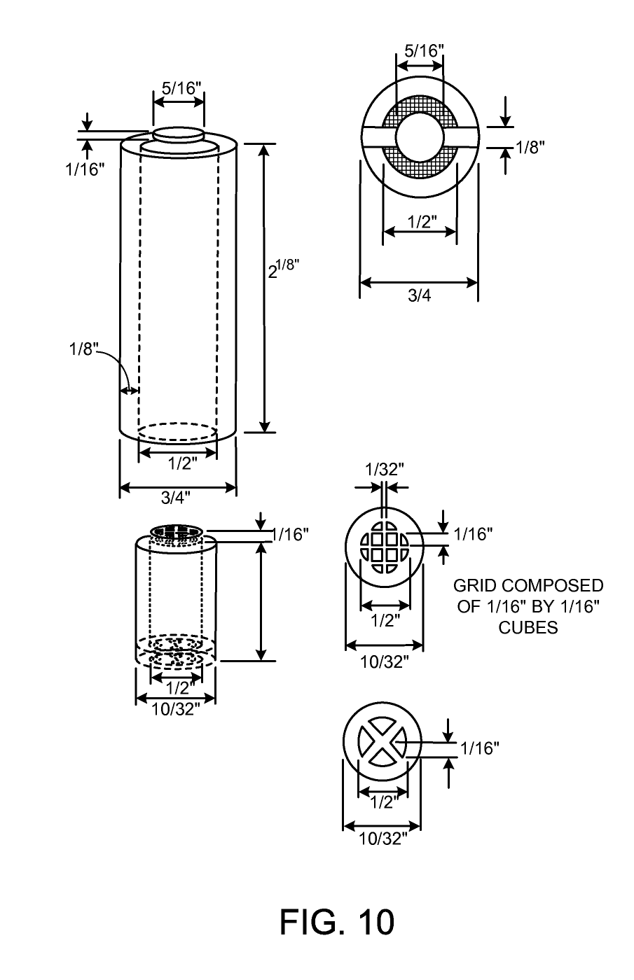

Especially, but not exclusively, in some embodiments in which intake is by inhalation or exhalation, minimum particle size is an important feature of the approach. The food aerosol particles are designed to be substantially delivered and deposited into the mouth, for example by the forces of gravity or inertial impaction, but to not be easily delivered and deposited substantially further into the respiratory tract, for example the trachea or lungs. Such particles (e.g., food particles) would thus possess a size larger than that which focuses penetration into the lungs (i.e., larger than about 10 microns). For example, breath-activated inhaler-like devices, such as the devices shown (in part or in whole) in FIGS. 5-17, generate an aerosol that would fairly easily follow the inhaled air toward the lungs were it not for the aerosol particles' larger size (and the delivery device's airflow-directing elements).

Especially, but not exclusively, in embodiments in which intake is by displacement of the subject or of the aerosol (e.g., with an aerosol cloud), maximum particle size is an important feature of the approach. Indeed, the aerosol cloud must remain suspended in air for at least a brief time so that displacement into the mouth can occur. Thus the particles must not be so large such that they rapidly settle from the air. This will greatly depend on the force(s) and/or mechanism(s) by which the particles are held in the air (e.g., by “natural” forces alone, such as inertia, diffusion, etc., or by additional forces, such as an impeller, air currents, convection, etc.). Accordingly, in some embodiments, the particles should be less than about 500 microns under typical suspension forces and mechanisms. For example, ultrasound sources in liquid food products can produce a standing aerosol cloud that, so long as convection is minimal, balances gravity, diffusion, inertial impaction, and other forces, to stay suspended in the air.

The specific parameters of the apparatus and intake method will in part determine whether the subject is “inhaling”/“exhaling” or “eating”/“sipping” when intake of the aerosol occurs. This generally corresponds to (1) whether the aerosol is entering the subject's mouth and/or throat via breathed air (physiologically, while the epiglottis is directing the air into the trachea toward the lungs) or whether the aerosol is entering the subject's mouth by another method (such as displacement of the aerosol or of the subject), and (2) whether the subject's maneuver or expectation is equivalent to the consumption of a food product to be (eventually) swallowed (e.g., as with the use of a drinking straw while drawing fluid into the mouth, before swallowing; physiologically, while the epiglottis is blocking passage to the trachea). In any case, it should be further noted that the food product, after deposition in the mouth, may be eventually swallowed and consumed essentially as any other typical food product.

In some cases, the aerosol may be carried via inhaled air that flows all the way to the lungs (for example, like the inhalation a smoker may have, which carries air and smoke through/from the cigarette, into the lungs). In some cases, the aerosol may be carried via “sucked” air that “stops” in the mouth (more like the approach used with a typical straw and beverage, or with cigars). (In some cases, elements of both approaches may be suitable.) This potential distinction may have important implications for a food aerosol device. For example, in the case in which the particles are carried by air that continues directly to the lungs, preventing deposition of particles too far into the respiratory tract is more dependent on the physical parameters of the particles, airflow, etc. In the case in which the particles are carried by air that is sucked into the mouth, it may be possible to carry particles of mean sizes, or with other properties, that would normally allow them to extend further than desirable into the respiratory system, but that, by virtue of the airflow “stopping” before the lungs, have them fall substantially into the mouth anyway.

In some embodiments of devices in which an aerosol is generated by inhalation, e.g. the devices shown in FIGS. 5, 6, and 20, relatively dry, solid food powders of appropriate size can be used as the food product. Preliminary tests have shown that the water-solubility of the dry powders used plays a role in the taste and potential coughing reflex resulting from intake of the aerosolized food product. Powders of particles that tend to be more rapidly water-soluble, such as ground chocolate bars, or certain chocolate-based powders, give rise to a generally pleasing reaction upon contact of the particles with the tongue and other surfaces within the mouth. In the case of ground chocolate bars, for example, the effect is in some cases similar to that of sensing chocolate melt very rapidly in one's mouth. Particles that are less water-soluble, such as certain ground-cocoa-based powder products, tend to be considered harsher and more likely to elicit less pleasurable reactions, such as a dry-mouth sensation or coughing. However, in some instances, a combination of both kinds of powders, in varying proportions, provides interesting flavor complexity.

### Food Products, Including Aerosol Powders

By designing a food form that can be aerosolized (particles much larger than 500 microns fall quickly out of the air unless supported by an external force) and yet has sufficiently large particles (greater than approximately 1, 2, 3, 4, 5, 10, 15 or 20 microns) such that few or no particles enter the lungs on inspiration, our technology results in deposition and delivery into the mouth. Ideally, the particles would be designed (sized) such that, for example, at least about 50%, at least about 60%, at least about 70%, at least about 80%, at least about 90%, at least about 95%, at least about 97%, or at least about 99% of the particles deposit in the mouth and do not extend further into the respiratory tract. The design of the particles should also take into consideration reducing any tendency to cough, gag, or otherwise react unfavorably to the aerosol.

Dry powder particles can be created through a number of different methods. Initially, the food product may be dehydrated. Alternatively or in addition, where the food is a more malleable or liquid based food, the food may be frozen first to facilitate subsequent grinding or chopping. The food product may subsequently be ground to form particles (e.g., food particles) of the appropriate size. Grinding of the food products can be performed by use of a mortar and pestle. Alternatively or in addition, food products may be chopped, for example using a mechanical or electrical grinder, knives, etc. The resulting ground or chopped particles (e.g., food particles) can subsequently be filtered through sieves (for example by hand, using an electrical or mechanical sieve shaker, by an air classification system, by a screening system, etc.) to achieve the appropriate particle size. Another approach is to use a powder mill that grinds down larger particles into pre-defined sizes. Spray drying, in which a mixture of water and the material to be dried is forced through a nozzle into a high-temperature drum, instantly evaporating the water droplets clinging to the material, may also be utilized. These methods, in addition to others, would allow for the creation of specifically sized particles capable of being aerosolized, but large enough not to pass easily through the mouth and throat and continue into the respiratory tract.

These dry powder particles could be created from a single food or ingredient, such as chocolate, coffee, or truffles, or from a combination of foods or ingredients, such as combinations representative of an entire dish or meal (e.g., mixed fruits or meat and potatoes). In the case of chocolate, chocolate bars, chocolate powder, cocoa powder, and other forms and varieties of foods derived from the cocoa plant may be used. In addition, in some cases, spices and other (natural or artificial) flavorings may be used alone or in combination with such food ingredients to create other tastes or sensations (e.g., natural or artificial chocolate, raspberry, mango, mint, vanilla, cinnamon, caramel, and/or coffee flavors). Additionally, the apparatus may contain a single dose of food product or multiple doses/portions of the food product. In addition, they may be made from largely liquid products, for example by extracting dissolved solids or using other solid components. In some embodiments, flavors can be experienced while using less of the actual product compared to normal ingestion. In addition, by mixing different powders, new flavors can be created.

The food aerosol may also be a liquid that is aerosolized, for example by an ultrasound source that is in communication with a liquid food product; or by a “spray” mechanism, similar to those for liquids and gases in spray cans (“aerosol cans”) or vaporizers. Such liquids may be prepared by a variety of processes such that they are or include a concentrate, additive, extract, or other form of a food product that in some way preserves or enhances, and can deliver, a taste.

A liquid aerosol may also be generated by an ultrasonic device, such as vibrating piezo-electric discs placed within a container of liquid food product.

Depending on the food product(s) and device(s) used, the food product may be stored and/or contained in the form of a tablet or pill, in a blister pack, within a capsule, as simply a powder in a jar-like container, and/or in a tray, box, container, thermos, bottle, etc.

In some embodiments, it is possible to deliver odors using appropriately designed and appropriately sized particles, which may be utilized independently or in addition to embodiments described herein, i.e., in addition to delivery of aerosolized food product so as to enhance the aesthetic experience.

Please note that “food product”, “aerosol”, “particle”, and other similar terms are used throughout this document, and though they may typically refer to small solid particles derived from foods, these terms may in some cases refer to any of the other food-derived products described herein.

### Other Potential Properties of the Aerosols

Humidity or other ambient atmospheric conditions, which may vary over time and/or space, can be used to trigger time- or location-dependent changes in the aerosol and/or in the sensory detection and transduction it initiates in the subject(s). These conditional triggers may lead the particles to take on different gustatory, olfactory, aerodynamic, chemical, physical, geometric, and/or other properties, which in turn may alter the taste, texture, color, size, aerosolizability, and/or other aspect of the particles. For example, humidity conditions during the preparation, storage, or consumption of a consumable aerosolizable product may be used to favorably change the particle size distribution.

The purpose of such conditional triggers is generally to create a more interesting and dynamic experience for the subject(s). The trigger may depend on reaching a threshold atmospheric condition (e.g., greater than 50% humidity), or a threshold associated with the subject. The atmospheric condition may change the aerosol particles themselves and/or may allow them to interact differently with the subject's sensory mechanisms. For example, in low-humidity air, an aerosol may take on one chemical/physical state, which gives it a first taste, and in high-humidity air, it may take on a different chemical/physical state, which gives it a second taste. As another example, an aerosolized aerosol may have initially no taste and/or odor, or an initial taste and/or odor reminiscent of a certain food product (which may, for example, be detected initially by a subject through the olfactory system, before intake of the aerosol through the mouth); and after the aerosol is taken through the mouth, the ambient environment of the mouth may trigger a change in the aerosol that gives it a taste and/or odor, or new taste and/or odor reminiscent of a different food product. Over time but while the food product is still in the mouth, it may continue to evolve, evoking different sensations for the subject. Mechanisms like these could be used to create the impression of sequentially eating different courses of a meal, such as an appetizer followed by a main course followed by dessert.

### Time Airborne/Suspension Time

Depending on the particular embodiment, the food product can be in aerosol form (airborne) for different durations. For example, in the case of an inhaler-based device, the food product typically remains airborne only for the time over which inhalation and intake occur, which may be, for example, up to about ½ second, up to about 1 second, up to about 3 seconds, up to about 5 seconds, up to about 8 seconds, up to about 10 seconds, up to about 15 seconds, or possibly greater time periods. Alternatively, where the particle delivery device operates by producing an aerosol cloud, the food product may remain suspended in the air for, for example, at least about 1, 2, 3, 4, 5, 6, 7, 8, 9, 10, 15, 20, 25, 30 or 60 seconds, or at least about 2, 5, 10, 15, 20, 30, 45, 60, 90, 120, or 180 minutes. Mechanical agitation of the aerosol cloud, for example, by convection, can serve to increase the time during which the aerosol cloud is suspended.

### Applications

Our apparatus can transform how food is experienced, allowing for an enhanced aesthetic experience of food. For example, the apparatus can allow subjects to experience food by exposing themselves to, for example, rooms filled with food clouds, immersive chambers and food straws. Indeed, businesses, restaurants and nightclubs could provide such “food experiences”.

In some embodiments, our technology can allow subjects to experience food by exposing themselves to aerosolized food via individual, hand-held, and/or portable devices. In some embodiments, our technology may be used in and/or associated with social contexts similar to candy eating or cigarette smoking. For example, some embodiments may be carried about and used at various points throughout the day, or used simultaneously by multiple users.

In various other embodiments, the technology can allow multiple subjects to have a communal experience while appreciating food aerosols, for example in embodiments in which a single aerosol-generating device is associated to multiple delivery devices, such as a pot-like container confining a liquid aerosol cloud that is delivered by breath actuation to multiple subjects each using independent mouthpiece devices with airflow-directing elements.

In addition, the apparatus can serve to provide nutrition to subjects either who are incapable of chewing or for whom delivery of food is not convenient. For example, the particle delivery apparatus may be useful for elderly or young children, for whom chewing or feeding is inconvenient. In addition, individuals with medical conditions that require them to be fed in particular ways (e.g., by a feeding tube or intravenously) may use certain embodiments of this invention as a way to experience and taste food again.

The apparatus can also serve to facilitate the intake of medication that may not be of a pleasurable taste. If used in conjunction with delivery of the medication, e.g. orally, the apparatus can provide an additional flavor that masks the flavor of the medication.

Alternatively, the proposed particle delivery apparatus may be used for weight control or addiction mitigation applications. For example, the particle delivery apparatus can allow for subjects to consume relatively small or negligible quantities of food products or certain unhealthy or addictive substances, and the exposure to the particles (e.g., food particles) via the apparatus may provide a sensation or satisfaction normally associated with the consumption of a larger quantity of the food or substance in question, thereby potentially satisfying hunger or addictive urges without the (potentially negative) consequences of actually consuming larger amounts of the substance(s). In some cases, this may be due to the higher surface area of the food product exposed to surfaces of the mouth, for example exposed to taste receptors, relative to the overall quantity (e.g., mass) of food product. Indeed, the particle delivery apparatus may form a basis for dieting, weight control and healthy eating programs (for example, by satisfying cravings for sweets, fatty foods, chocolate and caffeine) and addiction treatment (for example, by satisfying urges for alcohol, smoking, drugs but in much smaller, less harmful amounts).

In addition, the particle delivery apparatus may be used to improve quality of life, for example, with respect to individuals subject to special dietary restrictions. For example, the particle delivery apparatus may allow individuals who suffer from allergies (e.g., gluten allergy) or other conditions (e.g., lactose intolerance) that normally prevent them from consuming specific products to consume relatively small or negligible quantities of these products without triggering an allergic or physical reaction, while possibly providing a sensation or satisfaction normally associated with the consumption of a larger quantity of the food or substance in question.

Additionally, the particle delivery apparatus can serve as a means for taste-testing a number of items in a simple and efficient way. For example, a patron at a restaurant can taste test various dishes on the menu before making a selection. Additionally, chefs may use the particle delivery apparatus to test combinations of foods while cooking or designing a recipe. Similarly, the apparatus may serve as an aid in cooking lessons, as an international “dining” experience for a subject, as a way to teach children about food, etc.

Other useful applications of the particle delivery apparatus include, but are not limited to hunger relief (e.g., in the emergency conditions of a famine) and for animal feedings.

Terms and phrases including “inhalable”, “exhalable”, “inhalation”, “exhalation”, “breathable”, “respiration”, “respirable”, “aspiration”, “inspiration”, “expiration”, “sip”, “sipping”, “sucking”, and others, have been used throughout this disclosure—or could have been used, as exact or approximate equivalents—to describe certain aspects of the disclosed embodiments. It should be noted that the definitions of each of these terms and phrases must be understood based on context and other relevant information herein. The precise definitions as understood in certain fields (e.g., medicine, anatomy, mechanical engineering, etc.) may not always be applicable in part or in whole.

Terms including “mean” and “median” are used in this disclosure to describe, in general, an overall feature of a composition or apparatus (e.g., the distribution of sizes of consumable aerosol particles).

In addition, throughout this disclosure, “aerosol”, and similar terms (including singular and plural usages), are intended to refer to “a gaseous suspension of fine solid or liquid particles” (“aerosol” as defined in the American Heritage Dictionary online, 2011). For example, a dry powder that interacts with air and is carried via airflow toward the mouth, is considered to be within this definition. As another example, a plurality of liquid droplets substantially suspended in air as the result of ultrasonic agitation of a liquid, is also considered to be within this definition. Other examples of aerosols, and other relevant uses of such terms, would be evident to those skilled in the art; these examples and definition are therefore meant as clarification and in no way are intended to limit the scope or applicability of the terms as used herein.

### EXAMPLES

The following examples are expected to be illustrative and do not limit the scope of the claims.

### Example 1

To help determine an ideal particle size for food aerosolization from a single-actuation dry powder inhaler, mint powder samples, with approximate initial mean particle sizes of at least 140 microns, were utilized. A mortar and pestle was used to grind the dry mint powder. Mean particle size was reduced to as low as ˜11 microns, as determined using a HELOS-RODOS particle sizing system. Particles of different sizes were placed in separate size 3 capsules and tested in a hand-held inhaler.

Tests were made with samples of mint particles with approximate mean particle sizes of 140, 111, 72, 40, 18, and 11 microns. Capsules (each containing approximately 30-120 mg of mint) were placed in the aerosolizer and punctured, and the inhaler was actuated to release the particles into the air. A large fraction of the particles could be seen to fall within 5 seconds after release, though this fraction decreased with decreasing sample particle size. It was relatively high in tests with approximate mean particle sizes of 140, 111, and 72 microns, and relatively low in tests with approximate mean particle sizes of 40, 18, and 11 microns. Tests with approximate mean particle sizes of 18 and 11 microns produced fairly mist-like and uniform plumes, with fewer visually distinct particles.

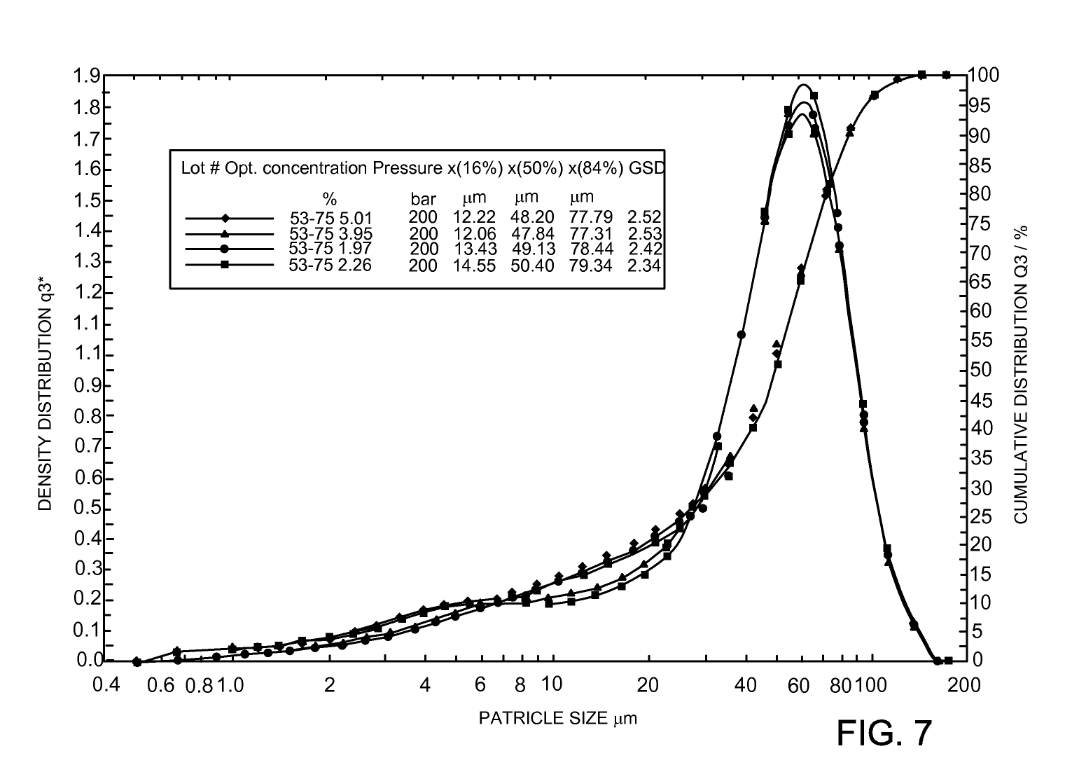

FIG. 7 shows the density distribution and cumulative distribution for four trials from the same sample. These data show that, for this particular sample, roughly 87% of the particles are larger than about 10 microns, and that roughly 79% of the particles are larger than about 20 microns. These findings demonstrate that a dehydrated food product (mint leaves) can be made into aerosolized particles substantially of a size (e.g. between at least 18 and 70 microns) that would typically deposit into the mouth upon inhalation.

In a sample of particles whose mean is approximately in this range, a small or negligible fraction of particles is able to enter into the throat and lungs and yet a considerable fraction of particles remains suspended for at least 5 seconds after a single inhaler actuation.

Clearly larger particle sizes could be aerosolized for at least as long with a larger aerosolization force or a more continued force of aerosolization, such as a continually or intermittently operating fan.

### Example 2

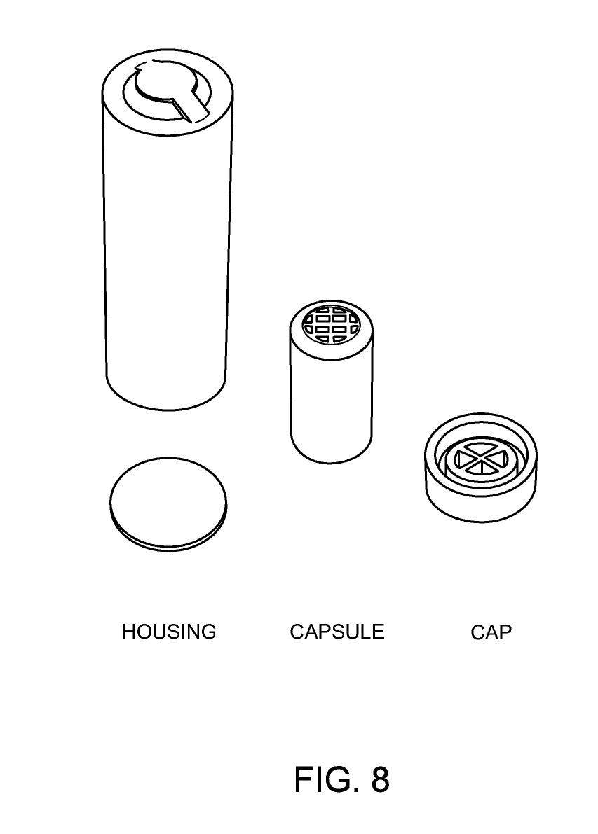

An aerosolized particle delivery device as depicted in FIGS. 8-10 was designed so as to deliver aerosolized chocolate. Chocolate was chopped into fine particles, which was subsequently screened by size. It was found that many readily available chocolates, when ground, remain dry enough to aerosolize in the delivery device described so long as care is taken not to handle the particles excessively, which causes them to quickly melt and fuse. The dryness of commercially available chocolate or cocoa powders makes such powders useful in producing a different aerosol taste experience, while enabling the powders to be far more stable (e.g. far less prone to melting). Using sieves, particular size ranges can be selected, and it was found that (likely among other size ranges), samples with a large number of particles with diameters roughly in the range of 125-180 microns are appropriate for strong taste and aerosolizability. It was also found that certain particles, even though of a size that should fall out of the air before reaching the deeper respiratory system (>10 microns), can cause a coughing reflex, even when of sizes reaching on the order of 100 microns or larger, but this is noticeably reduced with the airflow-directing mouthpiece element. It was also found that the water-solubility of the particles might play a role in the likelihood of eliciting a coughing reflex, and that water-solubility likely affects the taste, mouth-feel, and other gustatory aspects of the experience. For example, in some cases, the flavor of highly-water soluble particles is preferred, because the flavor is more rapidly appreciated and/or has a greater impact. Particles substantially larger than 180 microns are increasingly difficult to aerosolize and begin to taste like small pieces of chocolate simply dropped onto the tongue.

To simplify the filling procedure, it was determined that standard size 3 and size 4 capsules contain amounts of the chocolate powder appropriate for a single-inhalation “dose”. A standard manual capsule filling machine can thus be used to prepare a large number of such doses for transfer to the powder compartment of the delivery device.

### Example 3

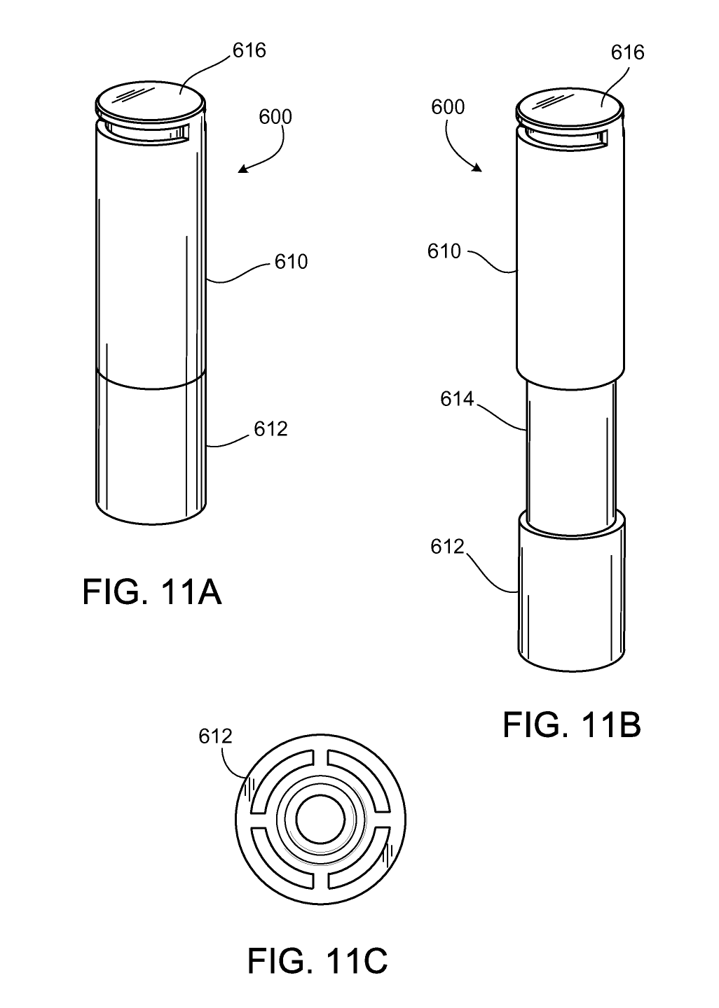

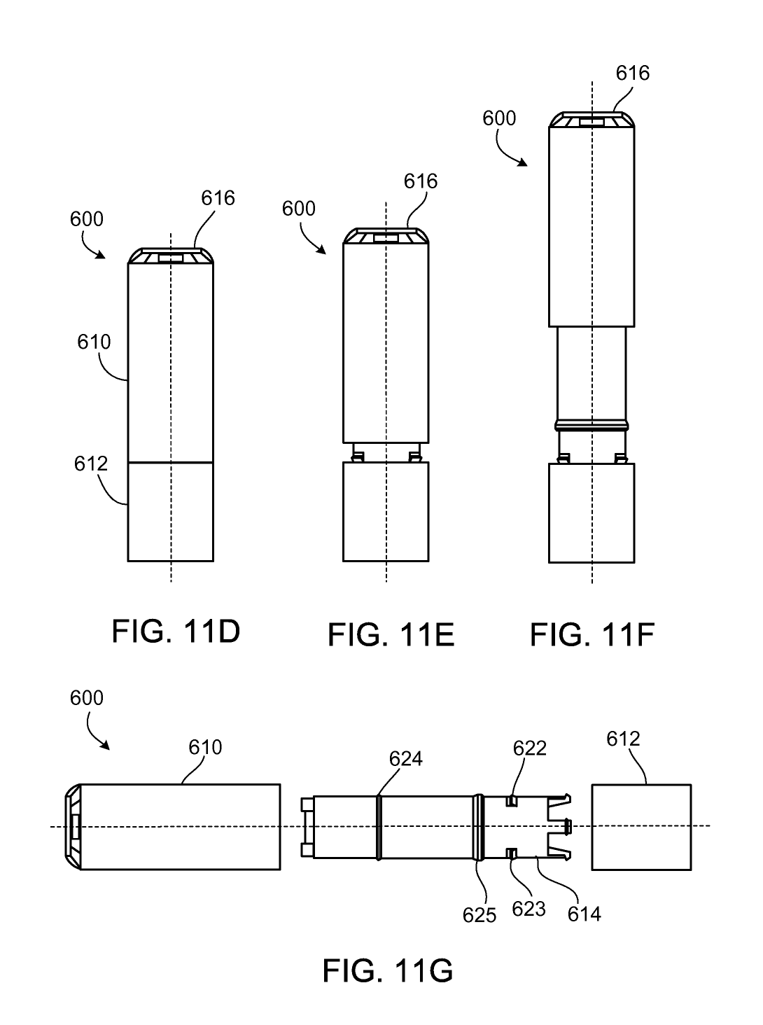

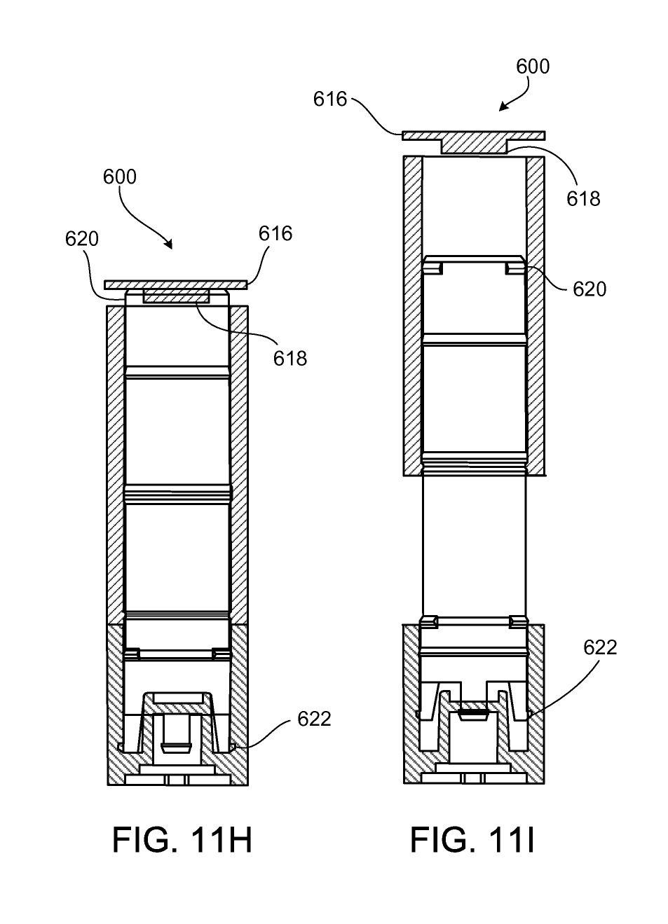

Another aerosolized particle delivery device 600 as depicted in FIGS. 11A and 11B was also designed to deliver aerosolized particles (e.g., food particles). Details of particle delivery device 600 are shown in FIGS. 11C-11G.

The particle delivery device 600 includes a mouthpiece 610, a cap 612, and a capsule 614 which can contain particles (e.g., food particles). The particle delivery device 600 differs from other embodiments (e.g., see FIGS. 2A-2G) in that the mouthpiece 610, the cap 612, and the capsule 614 have different relative overall lengths; disc sizes; and the contact between the capsule 614 and the mouthpiece 610, which allows the mouthpiece 610 to initially seal the top of the capsule 614. The mouthpiece 610 is shorter, and the capsule 614 is longer; the disc diameter is increased and equal to, or almost equal to, the mouthpiece outer diameter; and the capsule 614 is initially inside, and runs most or all of the length of the mouthpiece 610.

The mouthpiece 610 is generally cylindrical in shape. An additional cylinder 618 (see FIGS. 11H and 11I) below mouthpiece disc 616 and fits into the top of capsule 614 to seal a grating (not shown) through which aerosolized particles (e.g., food particles) are discharged when a user breathes in through the mouthpiece 610. The diameter of disc 616 is the same or nearly the same as the outer diameter of the mouthpiece 610. In one exemplary embodiment, the outer diameter of the mouthpiece 610 was 0.64 inches.

The capsule 614 is also generally cylindrical in shape. The end of the capsule 614 which is fitted into the mouthpiece 610 includes an annular ring 620 which extends axially outward from the rest of the capsule 614 (see FIGS. 11G-11I). The annular ring 620 is sized to fit around and engage the additional cylinder 618 under the mouthpiece disc 616, when the capsule 614 is fully inserted into the mouthpiece 610. The holes in the grating (not shown) atop the capsule 614 can thus be covered before activation of the device 600.

The capsule 614 includes snaps 622, 623 (see FIG. 11G-11I) at the bottom of the capsule 614 and allow the cap 612 to be either in an “open airflow” position (see FIGS. 11A, 11D, and 11H) or “closed airflow” position (see FIGS. 11B, 11F, and 20I). The capsule 614 also includes snaps 624, 625 near the top of the capsule are similar to the other two, in that the snaps 624 allow the mouthpiece 610 to be in a lower “closed airflow” position (see FIG. 11G-11I) and a higher “open airflow” position (see FIGS. 11A, 11D, and 11H).

Closed position snaps 622, 625 need to be undone to activate device 600, and were designed to be weaker than open position snaps 623, 624 which need to be permanent (difficult to undo). In some embodiments, the closure strength of the pairs of snaps can be the same or the open position snaps can be weaker than the closed position snaps.

Before filling, the mouthpiece 610 and the capsule 614 are snapped together. The cylindrical feature 618 underneath the mouthpiece disc 616 and the annular ring 620 on the capsule 614 align to seal the top of the capsule 614, keeping the particles (e.g., food particles) being loaded inside the capsule 614. The combined capsule-mouthpiece unit is then filled. The capsule 614 is then closed at the opposite end by the addition of the cap 612. The closures protect the particles (e.g., food particles) from the environment, and limit airflow (e.g., with the three pieces in the “closed” position, airflow to/from the capsule 614 is largely blocked on both sides). The device can be offered to consumers in this configuration (see, e.g., FIG. 11D).

For activation, the cap 610 is pulled downward (allowing airflow from the bottom) and then held by a snap (see, e.g., FIG. 11E). The mouthpiece 610 is similarly pulled upward and then held by a snap, introducing space between the top of the capsule 614 and the mouthpiece disc 616, allowing airflow through the top (see, e.g., FIG. 11F). This is the “open” position, and the device 600 can now be used.

The entire device can be disposed of, or recycled, after use. In the exemplary device 600, the mouthpiece 610, capsule 614, and cap 612 are configured for permanent attachment after assembly.

### Example 4

Other mouthpieces can be used with aerosolized particle delivery devices.

Curved Mouthpiece Outlets

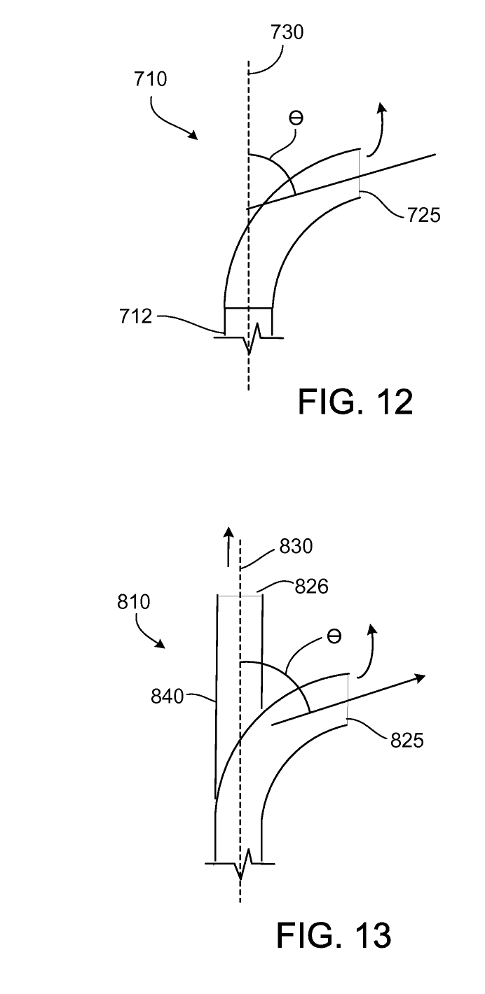

Another mouthpiece 710 as depicted in FIG. 12 is also designed to deliver aerosolized particles (e.g., food particles). Rather than including a mouthpiece disc used as an airflow directing element (e.g. see FIGS. 2A-2E, 4, 11A-11I), the mouthpiece 710 includes a mouthpiece outlet 725 oriented at an angle θ relative to a center axis 730 of the particle delivery device 712. In the illustrated embodiment, the walls of the outlet smoothly curve away from the center axis 730 of the particle delivery device 710 to the angle θ, which is approximately 70°. Due to the angular momentum of the particles as they travel through and exit the curved mouthpiece 710, it is expected they will travel towards a side of the mouth, the tongue, or the roof of the mouth, depending on which direction the mouthpiece 710 it is aimed. This allows the particles (e.g., food particles) to adequately coat a surface of the mouth without having an undesired trajectory which carries them to the back of the throat, potentially causing a cough reflex in the user.

In some embodiments, rather than a smooth curve, the mouthpiece outlet is at a sharp angle with respect to the particle delivery device center axis. For example, the mouthpiece outlet can abruptly bend or can even be perpendicular to the center axis of the particle delivery device

In some embodiments, the diameter of the mouthpiece outlet does not remain constant with respect to the axis of curvature of the mouthpiece. For example, the diameter of the mouthpiece outlet can widen and increase to reduce the airborne velocity of the particles (e.g., food particles). Conversely, the diameter can be decreased to accelerate the airborne velocity of the particles (e.g., food particles).

The mouthpiece can have a non-circular cross-section. Some mouthpieces have, for example, square, rectangular, or oval cross-sections.

In other embodiments, the angle at which the mouthpiece is positioned away from the particle delivery device center axis is any angle ranging between approximately 35°-180° (e.g., more than 45°, 55°, 65° and/or less than 115°, 105°, 95°).

Another mouthpiece 810 as depicted in FIG. 13 is also designed to deliver aerosolized particles (e.g., food particles). Rather than having a single mouthpiece outlet oriented at an angle relative to the particle delivery device center axis, the mouthpiece 810 has a primary mouthpiece outlet 825 oriented at an angle θ relative to a center axis 830 of the particle delivery device and a secondary mouthpiece outlet 826 that is in line with the center axis of the particle delivery device. Mouthpiece 810 also has a screen section 840 that is positioned just after the primary mouthpiece outlet 825 and is substantially tangential to the primary mouthpiece outlet 825. The screen section 840 has screen openings sized smaller than the aerosolized particles (e.g., food particles) such that the screen section 840 is expected to laterally deflect some or all of the aerosolized particles out of the primary mouthpiece outlet 825, while allowing air to pass through the screen section 840 and enter the mouth through the secondary mouthpiece outlet 826. The screen section 840 can include a rigid grating, flexible mesh, textile material, or porous filter such as a fabric. The screen section 840 can made of a wide range of materials. For example, the screen section 840 can be molded into the mouthpiece 810 using the same type of plastic material used to construct the mouthpiece 810. The screen section can alternatively be manufactured as an additional component made from a metallic material, a plastic material, a composite material, a paper based material, or a woven fiber material.

The characteristics of the secondary mouthpiece outlet 826 can be altered in the same manners as the mouthpiece outlet 725 oriented at an angle, as discussed above and in FIG. 12.

Lateral Mouthpiece Outlets

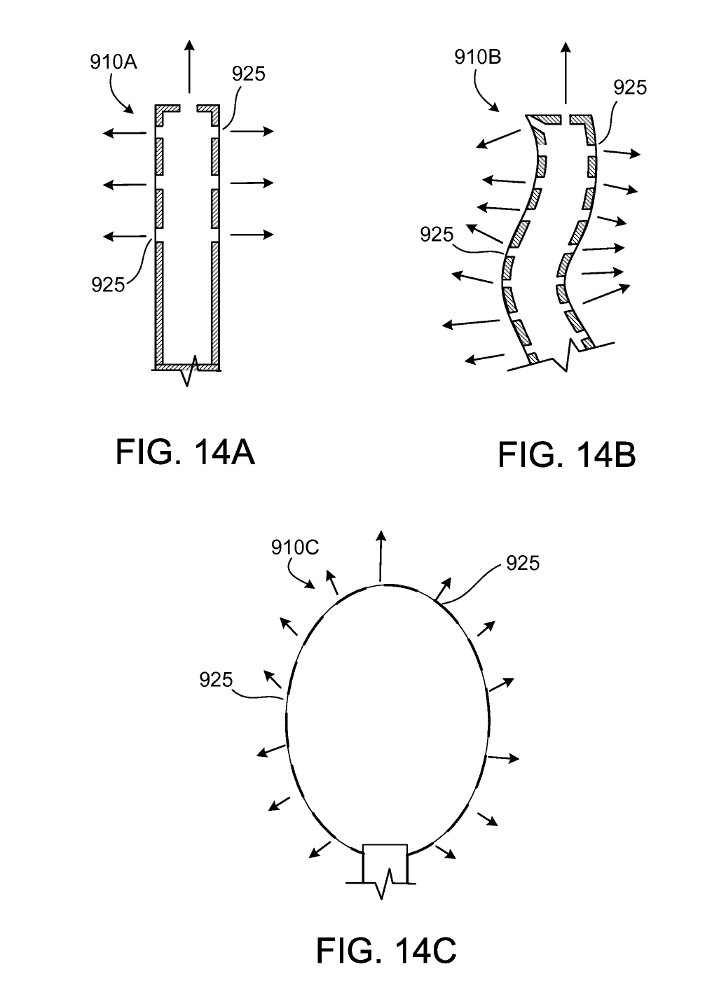

Other mouthpieces as depicted in FIGS. 14A-14C are designed to deliver aerosolized particles (e.g., food particles). Rather than having generally tubular shaped mouthpieces with outlets only at the mouthpiece end, the mouthpieces 910A-910C have a plurality of mouthpiece lateral outlet orifices or openings 925 including, for example, outlets on substantially all of the surfaces that are inserted in the mouth during use. By having many mouthpiece outlet openings 925 on both the surfaces that directly face the back of the mouth and throat area (e.g., inhaler orifices) as well as mouthpiece outlet openings that face the other areas of the mouth such as the sides, tongue or roof of the mouth (e.g., particle dispensing orifices), particles (e.g., food particles) are expected to exit the mouthpiece through all of the mouthpiece outlet openings 925 in a slower, more controlled manner and will not be throttled towards the throat which can induce a coughing reflex. As shown in FIGS. 14A-14C, the general shapes of such mouthpieces include a simple cylinder or rectangle 910A, a curvy cylinder or rectangle 910B, and a bubble shaped disk or sphere 910C.

In other embodiments, the mouthpiece can be many other novelty shapes as long as the majority of the mouthpiece surfaces do not face the back of the throat. For example the mouthpiece can be shaped like a star, a diamond, a polygonal, a chocolate bar, or something else, for example that is representative of the particles (e.g., food particles) inhaled.

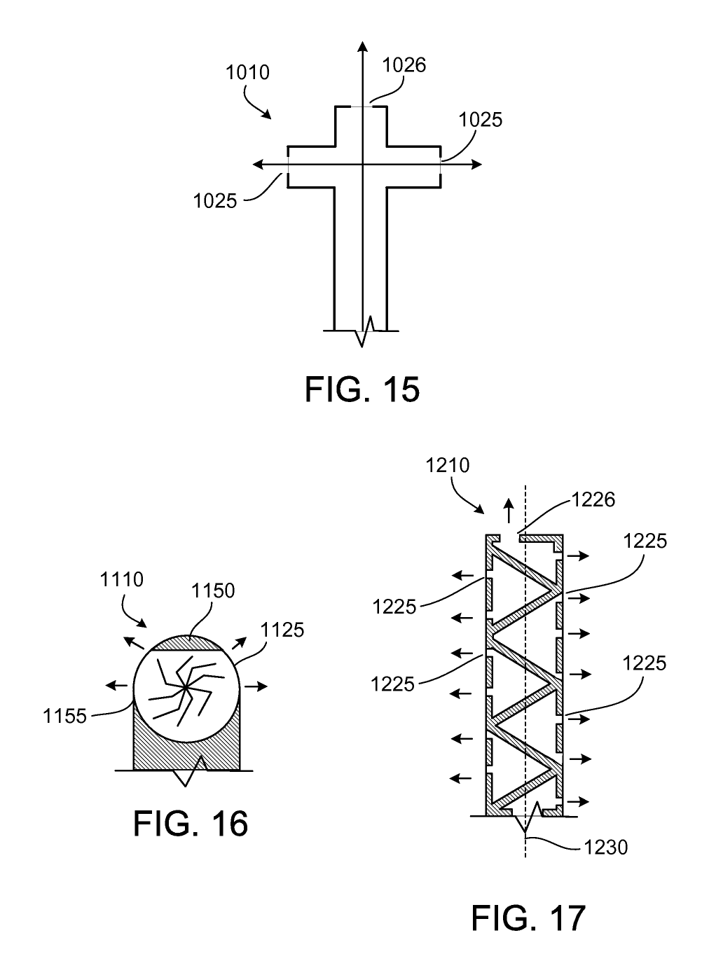

Another mouthpiece as depicted in FIG. 15 is designed to deliver aerosolized particles (e.g., food particles). Mouthpiece 1010 has a substantially straight secondary mouthpiece outlet (e.g., inhaler orifice) 1026 with two primary mouthpiece outlets (e.g., dispensing orifices) 1025 that exit the secondary mouthpiece outlet in a substantially perpendicular to the particle delivery device center axis and point in opposite directions.

In another embodiment, the mouthpiece can include more than two primary mouthpiece outlets. For example, a mouthpiece can include 3, 4, 5, 6, or more primary mouthpiece outlets. Also, the primary mouthpiece outlets do not need to be spaced evenly around the secondary mouthpiece outlet. For example, all of the primary mouthpieces can be aimed towards the tongue, roof of the mouth, or one of the sides.

Radial Fan Mouthpieces

Another mouthpiece as depicted in FIG. 16 is designed to deliver aerosolized particles (e.g., food particles). Mouthpiece 1110 includes fan blades 1150 positioned to rotate about an axis that is parallel to the center axis 1130 of the mouthpiece 1110. As shown, the fan can be positioned to rotate about the center axis 1130 of the mouthpiece 1110. Mouthpiece 1110 can be configured so that when a user sucks on the mouthpiece 1110, the fan blade 1150 rotates. The rotation of the fan blade 1150 can retard the flow entering the user's mouth to prevent aerosolized particles (e.g., food particles) from accelerating to the back of the user's throat and causing a coughing reflex. Additionally or alternatively, the fan blade 1150 can be configured (e.g., by choice of blade dimensions and angles) to propel the aerosolized particles (e.g., food particles) radially outward as they exit the mouthpiece, so that the majority of the aerosolized particles (e.g., food particles) are expected to coat the sides of the mouth instead of accelerating to the back of the throat which can cause a coughing reflex. For example, a turbine-style fan blade is expected to produce rotational movement in the fan blade while propelling aerosolized particles (e.g., food particles) radially outward.

In some embodiments, the mouthpiece can be formed without outlets in the distal end of mouthpiece to limit travel of particles towards the back of the throat.

In other embodiments, the blade 1150 may be fixed in position so that it does not rotate, but configured (e.g., by the angle of the blades still propels aerosolized particles (e.g., food particles) radially outward.

In other embodiments, the rotation of the fan blade can create a form of audible or visual stimulation. For example, the fan blade can include light emitting elements that rotate with the fan blade, or a mechanism to generate a sound that corresponds with the fan blade's rotation.

Mouthpieces with Interior Flow Partitions

Another mouthpiece as depicted in FIG. 17 is designed to deliver aerosolized particles (e.g., food particles). Mouthpiece 1210 includes a flow directing partition 1250 configured to deflect aerosolized particles (e.g., food particles) outward through multiple side mouthpiece outlet openings (e.g., dispensing orifices) 1225. Flow directing mouthpiece partition device 1250 includes a generally zig-zag or corkscrew shape to deflect particles (e.g., food particles) outwardly, perpendicular to the center axis of the device. Mouthpiece 1210 also includes a secondary mouthpiece outlet opening (e.g., inhaler orifice) 1226 that fluid drawn through the mouthpiece may also enter the mouth through. As there are many more side mouthpiece outlet openings 1225 than the single secondary mouthpiece outlet opening 1226, the majority of the aerosolized particles (e.g., food particles) are expected to coat the sides of the mouth instead of accelerating to the back of the throat potentially which can cause a coughing reflex.

In other embodiments, a flow directing partition can shaped differently. For example, a partition along the center axis of the mouthpiece and smaller partitions are at an angle to the center axis of the mouthpiece (for example are perpendicular to the center axis of the mouthpiece) to coincide with the mouthpiece outlet openings.

Mouthpieces with Interior Moving Parts to Mechanically Control Flow

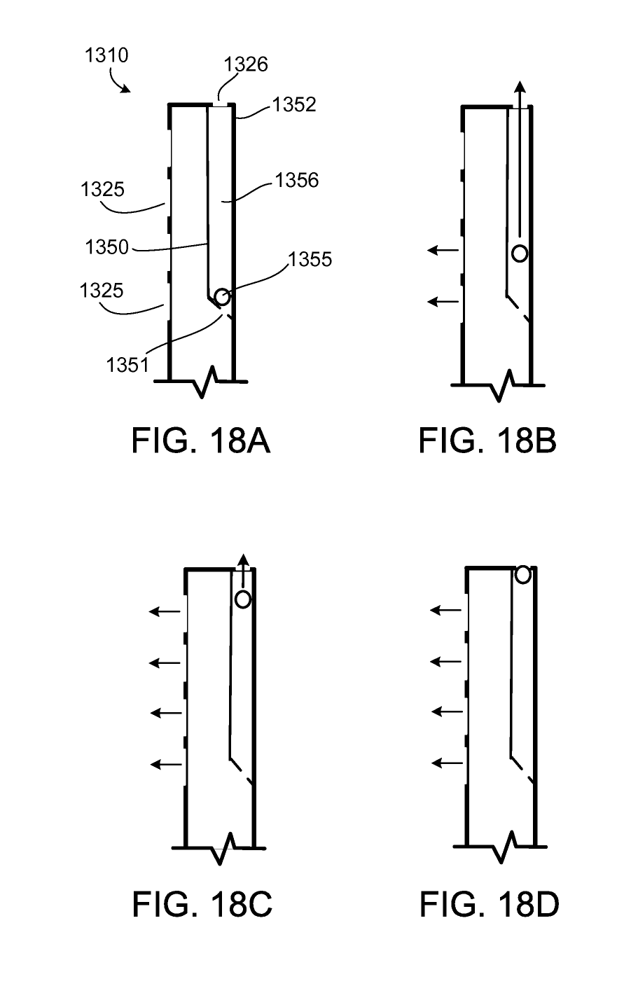

Another mouthpiece as depicted in FIG. 18A-18D is designed to deliver aerosolized particles (e.g., food particles). Mouthpiece 1310 includes a plurality of mouthpiece outlet openings (e.g., dispensing orifices) 1325 along the side of the mouthpiece outlet 1310 and a secondary mouthpiece outlet (e.g., inhaler orifice) 1326 positioned at an end of the mouthpiece. The mouthpiece 1310 further comprises a rigid partition 1350 that acts as a translational track 1356 for a moveable member 1355 that can roll or move throughout the mouthpiece 1310 within the translational track 1356. In the illustrated embodiment, the moveable member 1355 is a ball. However, in some embodiments, the moveable member 1355 is another shape such as, for example, a cube or a rectangular block.

A track air inlet 1351 is positioned at the distal end of the translational track 1356. When a user sucks on the end of the mouthpiece, particles (e.g., food particles) are expected to flow out through the mouthpiece outlet openings 1325. The pressure change within the mouthpiece 1310 will cause the movable member 1355 to translate (e.g., a ball will roll) towards the secondary mouthpiece outlet 1326 as depicted in FIGS. 18B and 18C. As depicted in FIG. 18D, once the ball 1355 reaches the secondary mouthpiece outlet 1326, the ball 1355 will block air from exiting the secondary mouthpiece outlet 1326 and particles (e.g., food particles) will continue to flow out of the mouthpiece outlet openings 1325 on the side of the mouthpiece 1310, substantially limiting the likelihood that particles (e.g., food particles) will accelerate to the back of the throat which could generate a coughing reflex in a user.

In some embodiments, the aerosolized particles disperse via outlets 1325 until the ball blocked the secondary outlet 1326, at which point the overall airflow stops. The resistances can be chosen such that the ball would block the secondary outlet 1326 at a point before the aerosol has substantially reached and exited 1326 (toward the throat) and instead has dispersed via 1325.

In some embodiments, a mouthpiece 1310 can be configured such that a dose of aerosolized particles (e.g., food particles) is measured to run out once the moveable member 1355 reaches the secondary mouthpiece outlet 1326.

In other embodiments, a mouthpiece can comprise multiple partitions to give the translational track a desired shape or path. For example, the track can be diagonal across a mouthpiece, it can follow a curved path, or some spiral.

As discussed above with respect to mouthpiece 1110, the translation of the ball 1355 can create a form of audible or visual stimulation. For example, a mouthpiece can comprise translating lights to correlate with the translation of the ball, or a sound that corresponds with the translation.

### Example 5

Other mouthpieces can be used with aerosolized particle delivery devices. Mouthpieces can include features or elements that permit aerosolization of particles (e.g., food particles) by an exhalation of a user. In some embodiments, a user exhales into a mouthpiece to aerosolize food particles and during or after the exhalation, the mouthpiece causes at least a portion of the aerosolized food particles to flow from the mouthpiece enter the user's mouth for deposition along and absorption within the user mouth.

For example, a mouthpiece can include an expanding balloon-like device into which the user can exhale (e.g., blow into as-if to blow up a balloon) to create pressure and cause expansion of the balloon. When the user releases the pressure, the expanded balloon-like device can contract and the force air containing aerosolized food particles back into the user's mouth.

In some examples, a mouthpiece can include spring-loaded closure devices (e.g., spring-loaded flaps) that are configured to be blown open when a user exhales into the mouthpiece. In some cases, when the user exhales into the mouthpiece, food particles are aerosolized and, when the user stops exhaling or releases pressure, the closure device can pivot or swing closed which can cause the air containing aerosolizes food particles to flow from the mouthpiece back into the user's mouth.

In some examples, a mouthpiece can include a first passage into which the user can exhale to deliver air to an aerosolizing reservoir. In the aerosolizing reservoir, the exhaled air can aerosolize the food particles to generate a body of air having aerosolized food particles. A second passage fluidly connects the aerosolizing reservoir back to the mouth so that the air forced into the mouthpiece during exhalation can carry the aerosolized air back into the mouth for deposition along and absorption within the user mouth.

### Example 6

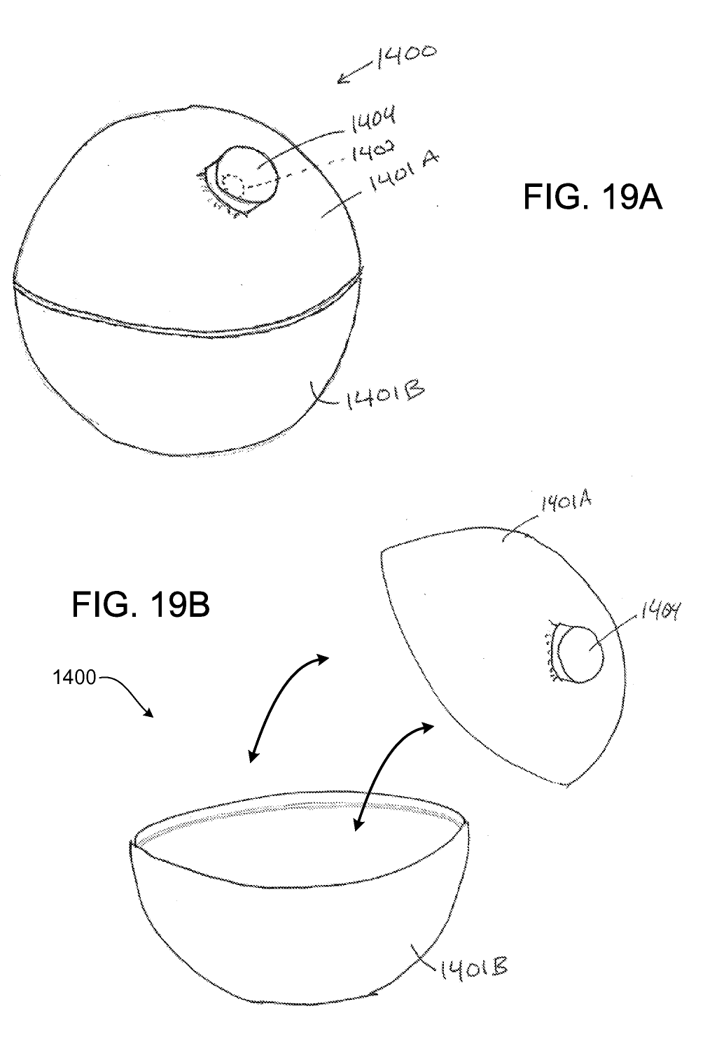

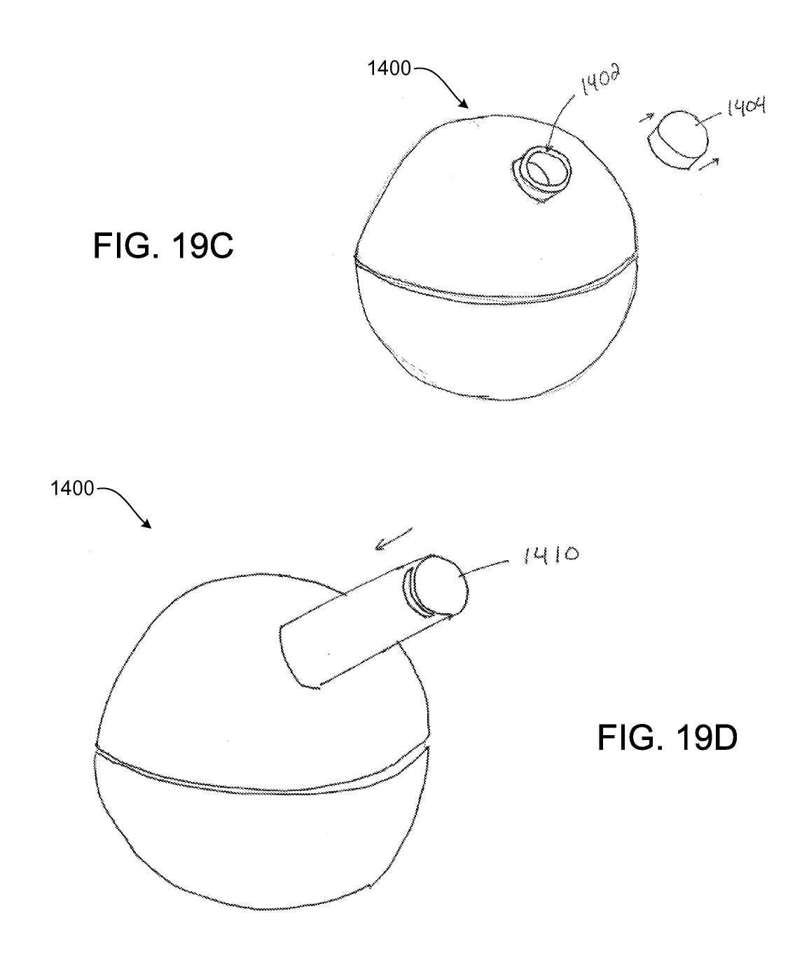

Other methods and devices can be used for delivering aerosolized particles. For example, in some cases, aerosolization can occur by shaking fine particles causing them to become airborne and aerosolize. In some embodiments, referring to FIGS. 19A-19D a container (e.g., a semi-spherical container) 1400 made of a structurally suitable material (e.g., metal) is formed into two semi-hemispherical halves 1401A, 1401B that can be connected to and decoupled from one another to enclose particles (e.g., food particles). The two container halves 1401A, 1401B can be opened and an amount of food particles (e.g., about 10 grams) can be deposited into one of the halves (shown in FIG. 19B), and the two container halves 1401A, 1401B can be connected to one another enclosing the food particles. The size of the container 1400 and the amount of food particles deposited therein are typically selected so that a large majority of the container 1400 is empty when the halves 1401A, 1401B are connected. In some cases, one of the halves 1401B can have flat surface so that the container 1400 can be set and supported on a flat surface (i.e., without rolling). A small opening 1402 and an associated removable cap 1404 can be arranged along the other container half 1401A. The cap 1404 and opening 1402 are configured so that if the cap 1404 is removed, a mouthpiece 1410 (e.g., one or more of the mouthpieces described herein) can be attached to (e.g., slipped or screwed into) the opening 1402.

To dispense an aerosol of enclosed food particles from the container 1400, the container 1400 can be shaken (e.g., using hands or another device) to excite the food particles and create an aerosolized cloud within the container 1400. Referring to FIGS. 19C and 19D, the cap 1404 is then removed and the user can ingest a portion of the aerosolized food particles. For example, one of the mouthpieces described herein can be attached to the opening and the user can inhale (suck in) or exhale into the mouthpiece to cause the food particles to entire his or her mouth. In some cases, the user inhales or exhales directly from the opening.

Using containers in this manner can permit a user to use his or her own mouthpiece and connect it to different containers in order to sample various types of food particles using the same mouthpiece.

What is claimed is:

1. A particle delivery apparatus comprising:
an aerosol delivery device for discharge of aerosolized particles to a user; the aerosol delivery device including:
a mouthpiece defining an inlet, an outlet, and a fluid flow passage extending therebetween, the inlet configured to direct the aerosolized particles along the flow passage in a direction generally toward a user's throat; and
a deflector disposed along the flow passage, the deflector configured to redirect at least a portion of the aerosolized particles along the flow passage from the inlet toward the outlet, the outlet positioned to direct aerosolized particles from the flow passage toward at least one side of a user's mouth.

2. The particle delivery apparatus of claim 1, wherein the deflector comprises a curved portion.

3. The particle delivery apparatus of claim 2, wherein the curved portion is configured to redirect at least a portion of the aerosolized particles by about 90 degrees.

4. The particle delivery apparatus of claim 2, wherein the curved portion comprises a substantially continuous curve extending from the inlet to the outlet.

5. The particle delivery apparatus of claim 1, wherein the deflector is configured to redirect aerosolized particles about 90 degrees along the flow passage.

6. The particle delivery apparatus of claim 1, wherein the mouthpiece further defines an inhaler orifice in fluid communication with the fluid flow passage, the inhaler orifice positioned to direct at least a portion of inhaled fluid toward the user's throat.

7. The particle delivery apparatus of claim 6, wherein the inlet defines a first plane, the outlet defines a second plane, the inhaler orifice defines a third plane, and at least one of the first and third plane is nonparallel to the second plane.

8. The particle delivery apparatus of claim 7, wherein at least one of the first and third plane is substantially perpendicular to the second plane.

9. The particle delivery apparatus of claim 7, wherein the first plane is substantially parallel to the third plane.

10. The particle delivery apparatus of claim 6, wherein the pressure drop of fluid flowing between the inlet and the inhaler orifice is greater than the pressure drop of fluid flowing between the inlet and the outlet.

11. The particle delivery apparatus of claim 6, wherein the minimum cross-sectional open area of the flow passage between the inlet and the inhaler orifice is less than the minimum cross-sectional open area of the flow passage between the inlet and the outlet.

12. The particle delivery apparatus of claim 6, wherein the cross-sectional open area of the inhaler orifice is less than the cross-sectional open area of the outlet.

13. The particle delivery apparatus of claim 12, wherein the ratio of the cross-sectional open area of the inhaler orifice to the cross-sectional open area of the outlet is between about 0.1 to about 0.9.

14. The particle delivery apparatus of claim 6, wherein the outlet comprises a plurality of orifices defined by the mouthpiece, the plurality of orifices disposed between the inlet and the inhaler orifice.

15. The particle delivery apparatus of claim 14, wherein at least some of the plurality of orifices are spaced along an axis substantially parallel to an axis defined by the inlet and the inhaler orifice.

16. The particle delivery apparatus of claim 14, wherein at least some of the plurality of orifices are spaced about a circumference of the mouthpiece.

17. The particle delivery apparatus of claim 6, wherein the flow passage is elongate in a direction between the inlet and the inhaler orifice.

18. The particle delivery apparatus of claim 17, wherein the outlet comprises a plurality of orifices axially spaced from one another along an axis substantially parallel to an axis defined by the inlet and the inhaler orifice.

19. The particle delivery apparatus of claim 6, wherein the deflector is movable to restrict the flow of fluid through the inhaler orifice.

20. The particle delivery apparatus of claim 19, wherein the deflector is movable by fluid flowing through the flow passage.

21. The particle delivery apparatus of claim 19, wherein the deflector comprises a pinwheel disposed along the flow passage, the pinwheel rotatable about an axis substantially parallel to an axis defined by the inlet and the inhaler orifice.

22. The particle delivery apparatus of claim 19, further comprising a track disposed substantially within the mouthpiece, along the flow passage, wherein the deflector is configured to move along the track.

23. The particle delivery apparatus of claim 22, wherein at least a portion of the track extends in a direction parallel to an axis defined by the inlet and the inhaler orifice.

24. The particle delivery apparatus of claim 6, wherein the deflector and the mouthpiece define a substantially helical flow path along at least a portion of the flow passage between the inlet and the inhaler orifice.

25. The particle delivery apparatus of claim 24, wherein the outlet is defined along a portion of the mouthpiece defining the helical flow path.

26. A particle delivery apparatus comprising:
an aerosol delivery device for discharge of aerosolized particles to a user; the aerosol delivery device including:
a mouthpiece defining an inlet, an inhaler orifice, a fluid flow passage extending therebetween, and an outlet in fluid communication with the flow passage, the inlet configured to direct fluid along the flow passage in a direction generally toward a user's throat; and
a first reservoir defining a first outlet directed generally toward at least one side of a user's mouth, the first reservoir configured to support aerosolized particles, and the first reservoir in fluid communication with the flow passage such that fluid flowing through the flow passage displaces at least a portion of the aerosolized particles through the first outlet.

27. The particle delivery apparatus of claim 26, wherein the reservoir is releasably coupled to the mouthpiece.

28. The particle delivery apparatus of claim 27, wherein the reservoir is disposable.

29. The particle delivery apparatus of claim 26, wherein the pressure drop of fluid flowing through the first reservoir is less than the pressure drop of fluid flowing through the flow passage.

30. The particle delivery apparatus of claim 26, wherein the outlet comprises a plurality of orifices.

31. The particle delivery apparatus of claim 26, further comprising a second reservoir defining a second outlet directed generally toward at least one side of a user's mouth, the second reservoir configured to support aerosolized particles, and the second reservoir in fluid communication with the flow passage such that fluid flowing through the flow passage displaces at least a portion of the aerosolized particles through the second outlet.

32. The particle delivery apparatus of claim 31, wherein the first outlet and the second outlet define an axis substantially perpendicular to an axis defined by the inlet and the inhaler orifice.

33. A method of delivering particles, the method comprising:
receiving aerosolized particles in a mouthpiece defining an inlet, an outlet, and a fluid flow passage extending therebetween;
directing the aerosolized particles along the flow passage from the inlet in a direction generally toward a user's throat;
redirecting at least a portion of the aerosolized particles from the direction generally toward a user's throat generally toward the outlet, the outlet positioned to direct aerosolized particles from the flow passage toward at least one side of a user's mouth.

34. The method of claim 33, further comprising directing inhaled fluid toward an inhaler orifice defined by the mouthpiece, the inhaler orifice positioned to direct at least a portion of inhaled fluid toward the user's throat.

35. The method of claim 34, wherein redirecting at least a portion of the aerosolized particles comprises restricting flow to the inhaler orifice.

36. The method of claim 33, wherein redirecting at least a portion of the aerosolized particles comprises moving a deflector disposed in the flow passage.
